(19) United States
(12) Patent Application Publication
(10) Pub. No.: US 2024/0401073 A1
(43) Pub. Date: Dec. 5, 2024

(54) ALS INHIBITOR HERBICIDE TOLERANT BETA VULGARIS MUTANTS

(71) Applicant: KWS SAAT SE & Co. KGaA, Einbeck (DE)

(72) Inventors: Olaf CZARNECKI, Berlin (DE); Maik GERTZ, Hannover (DE); Jens Christoph LEIN, Göttingen (DE); David WURBS, Einbeck (DE)

(73) Assignee: KWS SAAT SE & Co. KGaA, Einbeck (DE)

(21) Appl. No.: 18/699,357

(22) PCT Filed: Oct. 14, 2022
(86) PCT No.: PCT/EP2022/078627

§ 371 Date: Apr. 8, 2024

(30) Foreign Application Priority Data
Oct. 15, 2021 (EP) 21202922.7

(51) Int. Cl. C12N 15/82; A01N 43/50; A01N 43/54; A01N 43/653; A01N 43/90; A01N 47/36; A01P 13/02; C12N 9/10

(52) U.S. Cl. CPC C12N 15/8274; A01N 43/50; A01N 43/54; A01N 43/653; A01N 43/90; A01N 47/36; A01P 13/02; C12N 9/1022; C12Y 202/01006

(57) ABSTRACT

The present invention relates Beta vulgaris plants or plant parts having a mutated endogenous acetolactate synthase (ALS) protein comprising at position 371 an amino acid different than aspartate (D). Such plants are characterized in having increased tolerance to ALS inhibitor herbicides. The invention further relates to methods for generating such plants as well as methods for identifying such plants.

### FIELD OF THE INVENTION

The present invention relates to the technical field of crop protection. In particular, the invention relates to Beta vulgaris plants or parts thereof which are resistant or tolerant to acetolactate synthase (ALS) herbicides, as well as methods for generating and/or identifying such plants or plant parts and the use of such plants or plant parts in methods for controlling unwanted vegetation.

### BACKGROUND OF THE INVENTION

Acetolactate synthase (ALS) is an essential part of the branched chain amino acid biosynthesis pathways leading to leucine, isoleucine, and valine. ALS has been conserved across species and enzymes of bacteria, yeast and higher plants show substantial sequence similarities (Mazur et al. (1987): Isolation and characterization of plant genes-coding for acetolactate synthase, the target enzyme for 2 classes of herbicides. Plant Physiol. 85, 1110-1117). Interestingly, animals do not have the branched-chain amino acid pathway and therefore must ingest these amino acids in their diet. ALS is the first in a series of enzymes involved in the biosynthesis cycle for leucine and valine, which is located in chloroplasts. In Arabidopsis, AtALS forms a tetramer consisting of four identical subunits. Each subunit contains thiamine pyrophosphate (TPP) as a prosthetic group and catalyzes the formation of acetolactate from two molecules of pyruvate. Hereby, TPP reacts with one molecule of pyruvate to form hydroxyethyl-TPP and CO₂. The hydroxyethyl residue of TPP is subsequently transferred to the second molecule of pyruvate and acetolactate is formed. Then, acetolactate is further processed to valine and leucine. In parallel, ALS catalyzes threonine, and one molecule of pyruvate to 2-aceto-2-hydroxybutyrate which is further processed to isoleucine. ALS activity is feedback inhibited by leucine and valine, which bind synergistically on two separate domains of ALS to inhibit its activity.

ALS is the target enzyme for four classes of structurally unrelated herbicides (HRAC class B), the sulfonylureas, the sulfunylamino-carbonyl-triazolinones, the imidazolinones, and the triazolopyrimidines. These herbicide classes form the basis for more than fifty commercial herbicides that are globally used to protect essential rice, corn, wheat, and cotton crops. Sulfonylurea and imidazolinone bind to ALS and subsequently inhibit ALS activity. The sulfonyl group and the adjacent aromatic ring of the herbicides are situated at the entrance to a substrate channel leading to the active site of the enzyme with the rest of the molecule inserting into the channel (McCourt et al. (2006): Herbicide-binding sites revealed in the structure of plant acetohydroxyacid synthase. Proc. Natl. Acad. Sci. USA 103, 569-573). The substrate, e.g. pyruvate, cannot reach the enzymes active center anymore. This leads to an inhibition of the biosynthesis of leucine, valine and isoleucine and causes the herbicide effect.

Not long after the introduction of sulfonylureas and imidazolinones to the herbicide market, resistant weeds began to emerge (www.weedscience.org). These resistances are most commonly due to single point mutations resulting in amino acid substitutions. The most comprehensively characterized mutations are those of W574 (number indicate position based on the Arabidopsis thaliana protein sequence), which results in herbicide tolerance in several plants. The tryptophan residue serves to anchor both classes of herbicide to the enzyme and it is important for defining the shape of the active-site channel. Consequently, the commonly observed mutation of this residue to leucine changes the shape of the herbicide binding site and results in the loss of several interactions (Endo et al. (2013): Herbicide-resistant mutations in acetolactate synthase can reduce feedback inhibition and lead to accumulation of branched-chain amino acids. Food and Nutrition Sci. 4, 31233.).

A sulfonylurea and imidazolinone resistant sugar beet (Beta vulgaris L. spp. vulgaris) mutant has been identified many years ago by incubation of sugar beet cell cultures on SU-containing medium and subsequent callus induction and plant restoration. The mutant line carries the mutation W569L (corresponding to W574L in the Arabidopsis thaliana protein sequence), is tolerant to sulfonyl urea and imidazolinone, namely foramsulfuron and thiencarbazone-methyl, and serves as donor line to develop herbicide resistant varieties (WO 2012/049268). A weed control system using said herbicide resistance in sugar beets is commercialized since a few years under the brand CONVISO® SMART (www.convisosmart.com). Such herbicide resistant sugar beets carry the W569L homozygously in order to ensure maximum protection. WO 2014/091021 discloses a study with 22 different ALS inhibitor herbicides in sugar beets and shows that W569L in homozygous state conferred tolerance to all tested herbicides, only 7 herbicide composition were moderately toxic. In contrast thereto, sugar beet plants being heterozygous at position 569 have become only partially resistant towards several herbicidal compositions. 12 herbicide composition showed quite toxic effects and 7 were moderately toxic. Thus, for commercial application the use of W569L in homozygous state is clearly favourable.

However, the production of hybrid sugar beet seeds being homozygous for W569L requires enormous effort during breeding because the mutation needs to be introduced and maintained in both the maternal and paternal pool. Further, since any foreign, unwanted pollination by e.g. wild beets during hybrid production results in heterozygous hybrid seeds, high requirements for the seed quality control needs to be fulfilled in order to provide to the farmers only seeds carrying W569L homozygously. This is accompanied by additional costs and time for hybrid seed production.

Therefore, there is the need to further improve the ALS-inhibitor resistance in sugar beets and other cultivated forms of Beta vulgaris like fodder beets, red beets or Swiss chard.

### SUMMARY OF THE INVENTION

The present invention relates to the technical field of crop protection by using ALS (acetolactate synthase; also known as AHAS (acetohydroxyacid synthase; EC 2.2.1.6; formerly EC 4.1.3.18)) inhibitor herbicides against unwanted vegetation in areas of growing Beta vulgaris plants, such as sugar beet, fodder beet, swiss chard or red beet.

While a number of ALS mutations have been described in several weeds, it cannot be a priori expected that implementing such mutations in crop plants would be feasible and would convey the desired effect. Indeed, conferring ALS inhibitor resistance especially in crop plants, and in particular in Beta vulgaris which is known to be extremely sensitive to ALS inhibitor herbicides, requires the establishment of robust herbicide resistance. In this context, it is known that several identified ALS mutations only confer partial herbicide resistance, thereby precluding their use for commercial exploitation. Often, multiple mutations may even need to be combined in order to achieve agronomically useful and stable ALS inhibitor herbicide resistance. Furthermore, ALS mutations may only confer resistance to a select number of ALS inhibitor herbicides as a result of which options for weed control become limited. In addition, it has been described that ALS mutations may negatively impact growth characteristics and/or fertility, which in particular in crop plants is undesirable. Also, in order to confer robust herbicide resistance, ALS mutations may need to be present homozygously, which may pose a problem in breeding, in particular when generation of hybrids is desired.

The inventors have surprisingly found that mutating the Beta vulgaris ALS protein at position 371 confers robust ALS inhibitor herbicide resistance against a large variety of ALS inhibitor herbicides, while maintaining growth characteristics. Even more surprisingly, heterozygous ALS mutations were found to be equally suitable.

Accordingly, in an aspect, the invention relates to Beta vulgaris plants comprising mutations in the ALS gene where the aspartic acid at position 371 in the encoded ALS enzyme is substituted by another amino acid (preferably by glutamic acid). The present invention further provides novel non-transgenic Beta vulgaris donor lines that can be used to develop hybrid Beta vulgaris varieties resistant to ALS-inhibiting herbicides.

Amino acid substitution D371E bases on point mutation at nucleotide position 1141 of SEQ ID NO: 4, wherein T is replaced with an A. Consequently, the codon from nucleotide position 1139 to nucleotide position 1141, which is in wildtype GAT, is changed to GAA. Corresponding positions in the cDNA are nucleotide positions 1111-1113.

Another aspect of the present invention is providing a method of crop protection by using ALS inhibitor herbicides against unwanted vegetation in areas of growing Beta vulgaris plants comprising mutations in the ALS gene where the aspartic acid at position 371 in the encoded ALS enzyme is substituted by another amino acid (preferably by glutamic acid).

An advantage aspect of this invention is the existence of new and independent donors for resistance of ALS inhibiting herbicides that may help to circumvent undesired negative effects which may occur in known other donor lines caused e.g. by pleiotropic effects of the respective mutation or another negative linkage drag associated with the mutation.

Further, if for the production of hybrid sugar beets identical donors for both hybrid pools have been used it may happen that inbreeding depression can be observed, leading to a potential yield gap. The new donors of the present invention would allow that different donors for herbicide tolerance can be used in both pools, e.g., one pool with W569L mutation (see SEQ ID NOs: 10-12) and another according to the invention, such as with an ALS D371E mutation (see SEQ ID NOs: 1-3), thereby above problems may be solved.

Moreover, it may also be an option that the new donors are used to an independent product (Herbicide tolerant sugar beet lines) carrying only the mutation according to the invention, such as an ALS D371E mutation, heterozygous or homozygous.

In a further aspect, the invention relates to polynucleic acids encoding the mutated ALS protein according to the invention, as well as vectors comprising such polynucleic acids, and host cells comprising such polynucleic acids or vectors.

In a further aspect, the invention relates to methods for generating plants comprising the mutated ALS protein according to the invention, as well as methods for identifying plants comprising the mutated ALS protein according to the invention.

In a further aspect, the invention relates to the use of ALS inhibitor herbicides for controlling unwanted vegetation in crop areas, in which the crop plants comprise a mutated ALS protein according to the invention.

The invention is in particular captured by the appended claims, which are incorporated herein explicitly by reference.

### DETAILED DESCRIPTION OF THE INVENTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

Standard reference works setting forth the general principles of recombinant DNA technology include Molecular Cloning: A Laboratory Manual, 4^{th} ed., (Green and Sambrook et al., 2012, Cold Spring Harbor Laboratory Press); Current Protocols in Molecular Biology, ed. Ausubel et al., Greene Publishing and Wiley-Interscience, New York, 1992 (with periodic updates) (“Ausubel et al. 1992”); the series Methods in Enzymology (Academic Press, Inc.); Innis et al., PCR Protocols: A Guide to Methods and Applications, Academic Press: San Diego, 1990; PCR 2: A Practical Approach (M. J. MacPherson, B. D. Hames and G. R. Taylor eds. (1995); Harlow and Lane, eds. (1988) Antibodies, a Laboratory Manual; and Animal Cell Culture (R. I. Freshney, ed. (1987). General principles of microbiology are set forth, for example, in Davis, B. D. et al., Microbiology, 3rd edition, Harper & Row, publishers, Philadelphia, Pa. (1980).

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the following detailed description of the invention, reference is made to the accompanying drawings that form a part hereof, and in which are shown by way of illustration only of specific embodiments in which the invention may be practiced. It is to be understood that other embodiments may be utilised and structural or logical changes may be made without departing from the scope of the present invention. The following detailed description, therefore, is not to be taken in a limiting sense, and the scope of the present invention is defined by the appended claims.

Preferred statements (features) and embodiments of this invention are set herein below. Each statements and embodiments of the invention so defined may be combined with any other statement and/or embodiments unless clearly indicated to the contrary. In particular, any feature indicated as being preferred or advantageous may be combined with any other feature or features or statements indicated as being preferred or advantageous. Hereto, the present invention is in particular captured by any one or any combination of one or more of the below numbered aspects and embodiments 1 to 76, with any other statement and/or embodiments.
1. A Beta vulgaris plant, plant part, or plant population comprising, expressing, or capable of expressing a mutated endogenous acetolactate synthase (ALS) protein or a polynucleic acid encoding a mutated endogenous acetolactate synthase (ALS) protein comprising at position 371 an amino acid different than aspartic acid (D).
2. A Beta vulgaris plant, plant part, or plant population, optionally the Beta vulgaris plant, plant part, or plant population according to statement 1, comprising a mutated endogenous allele encoding an ALS protein comprising at position 371 an amino acid different than aspartic acid (D).
3. The Beta vulgaris plant, plant part, or plant population according to statement 1 or 2, wherein said ALS protein has a sequence which is at least 80% identical to SEQ ID NO: 3, preferably at least 90%, more preferably at least 95%, such as at least 98%.
4. The Beta vulgaris plant, plant part, or plant population according to any of statements 1 to 3, expressing or capable of expressing said ALS protein.
5. The Beta vulgaris plant, plant part, or plant population according to any of statements 1 to 4, wherein said ALS protein has ALS activity.
6. The Beta vulgaris plant, plant part, or plant population according to any of statements 1 to 5, wherein said ALS protein comprises glutamate (E) at position 371.
7. The Beta vulgaris plant, plant part, or plant population according to any of statements 1 to 6, wherein said ALS protein has a sequence of SEQ ID NO: 3.
8. The Beta vulgaris plant, plant part, or plant population according to any of statements 1 to 7, wherein said ALS is heterozygous.
9. The Beta vulgaris plant, plant part, or plant population according to any of statements 1 to 7, wherein said ALS is homozygous.
10. The Beta vulgaris plant, plant part, or plant population according to any of statements 1 to 9 which is tolerant to one or more ALS inhibitor herbicide.
11. The Beta vulgaris plant, plant part, or plant population according to any of statements 1 to 10 which is tolerant to one or more ALS inhibitor herbicide selected from (sulfon)amides such as sulfonylureas, sulfonylaminocarbonyltriazolinones, sulfonanilides, or triazolopyrimidines; imidazolinones; and pyrimidinyl(thio/oxy)benzoates, preferably selected from sulfonylureas, sulfonylaminocarbonyltriazolinones, imidazolinones, and pyrimidinyl(thio/oxy)benzoates.
12. The Beta vulgaris plant, plant part, or plant population according to statement 11, wherein said sulfonylurea is selected from one or more of
amidosulfuron [CAS RN 120923-37-7] (=A1-1);
azimsulfuron [CAS RN 120162-55-2] (=A1-2);
bensulfuron-methyl [CAS RN 83055-99-6] (=A1-3);
chlorimuron-ethyl [CAS RN 90982-32-4] (=A1-4);
chlorsulfuron [CAS RN 64902-72-3] (=A1-5);
cinosulfuron [CAS RN 94593-91-6] (=A1-6);
cyclosulfamuron [CAS RN 136849-15-5] (=A1-7);
ethametsulfuron-methyl [CAS RN 97780-06-8] (=A1-8);
ethoxysulfuron [CAS RN 126801-58-9] (=A1-9);
flazasulfuron [CAS RN 104040-78-0] (=A1-10);
flucetosulfuron [CAS RN 412928-75-7] (=A1-11);
flupyrsulfuron-methyl-sodium [CAS RN 144740-54-5] (=A1-12);
foramsulfuron [CAS RN 173159-57-4] (=A1-13);
halosulfuron-methyl [CAS RN 100784-20-1] (=A1-14);
imazosulfuron [CAS RN 122548-33-8] (=A1-15);
iodosulfuron-methyl-sodium [CAS RN 144550-36-7] (=A1-16);
mesosulfuron-methyl [CAS RN 208465-21-8] (=A1-17);
metsulfuron-methyl [CAS RN 74223-64-6] (=A1-18);
monosulfuron [CAS RN 155860-63-2] (=A1-19);
nicosulfuron [CAS RN 111991-09-4] (=A1-20);
orthosulfamuron [CAS RN 213464-77-8] (=A1-21);
oxasulfuron [CAS RN 144651-06-9] (=A1-22);
primisulfuron-methyl [CAS RN 86209-51-0] (=A1-23);
prosulfuron [CAS RN 94125-34-5] (=A1-24);
pyrazosulfuron-ethyl [CAS RN 93697-74-6] (=A1-25);
rimsulfuron [CAS RN 122931-48-0] (=A1-26);
sulfometuron-methyl [CAS RN 74222-97-2] (=A1-27);
sulfosulfuron [CAS RN 141776-32-1] (=A1-28);
thifensulfuron-methyl [CAS RN 79277-27-3] (=A1-29);
triasulfuron [CAS RN 82097-50-5] (=A1-30);
tribenuron-methyl [CAS RN 101200-48-0] (=A1-31);
trifloxysulfuron [CAS RN 145099-21-4] (sodium) (=A1-32);
triflusulfuron-methyl [CAS RN 126535-15-7] (=A1-33);
tritosulfuron [CAS RN 142469-14-5] (=A1-34);
NC-330 [CAS RN 104770-29-8] (=A1-35);
NC-620 [CAS RN 868680-84-6] (=A1-36);
TH-547 [CAS RN 570415-88-2] (=A1-37);
monosulfuron-methyl [CAS RN 175076-90-1] (=A1-38);
2-iodo-N-[(4-methoxy-6-methyl-1,3,5-triazinyl)carbamoyl]benzene-sulfonamide (=A1-39);
a compound of the general formula (I)

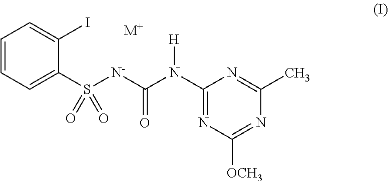

where M⁺ denotes the respective salt of the compound (1), i.e.
its lithium salt (=A1-40); its sodium salt (=A1-41); its potassium salt (=A1-42); its magnesium salt (=A1-43); its calcium salt (=A1-44); its ammonium salt (=A1-45); its methylammonium salt (=A1-46); its dimethylammonium salt (=A1-47); its tetramethylammonium salt (=A1-48); its ethylammonium salt (=A1-49); its diethylammonium salt (=A1-50); its tetraethylammonium salt (=A1-51); its propylammonium salt (=A1-52); its tetrapropylammonium salt (=A1-53); its isopropylammonium salt (=A1-54); its diisopropylammonium salt (=A1-55); its butylammonium salt (=A1-56); its tetrabutylammonium salt (=A1-57); its (2-hydroxyeth-1-yl)ammonium salt (=A1-58); its bis-N,N-(2-hydroxyeth-1-yl)ammonium salt (=A1-59); its tris-N,N,N-(2-hydroxyeth-1-yl)ammonium salt (=A1-60); its 1-phenylethylammonium salt (=A1-61); its 2-phenylethylammonium salt (=A1-62); its trimethylsulfonium salt (=A1-63); its trimethyloxonium salt (=A1-64); its pyridinium salt (=A1-65); its 2-methylpyridinium salt (=A1-66); its 4-methylpyridinium salt (=A1-67); its 2,4-dimethylpyridinium salt (=A1-68); its 2,6-dimethylpyridinium salt (=A1-69); its piperidinium salt (=A1-70); its imidazolium salt (=A1-71); its morpholinium salt (=A1-72); its 1,5-diazabicyclo[4.3.0]non-7-enium salt (=A1-73); its 1,8-diazabicyclo[5.4.0]undec-7-enium salt (=A1-74);
a compound of the formula (II) or salts thereof

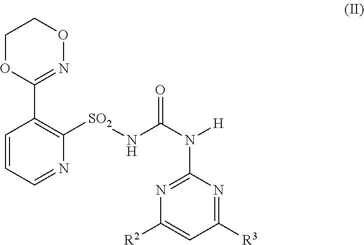

with R², and R³ having the meaning as defined in the below table

| Compound | R² | R³ |
|---|---|---|
| A1-75 | OCH₃ | OC₂H₅ |
| A1-76 | OCH₃ | CH₃ |
| A1-77 | OCH₃ | C₂H₅ |
| A1-78 | OCH₃ | CF₃ |
| A1-79 | OCH₃ | OCF₂H |
| A1-80 | OCH₃ | NHCH₃ |
| A1-81 | OCH₃ | N(CH₃)₂ |
| A1-82 | OCH₃ | Cl |
| A1-83 | OCH₃ | OCH₃ |
| A1-84 | OC₂H₅ | OC₂H₅ |
| A1-85 | OC₂H₅ | CH₃ |
| A1-86 | OC₂H₅ | C₂H₅ |

a compound of formula (III) (=A1-87), i.e. the sodium salt of compound (A1-83)

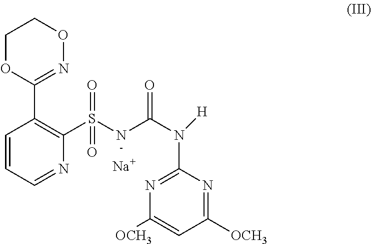 [SMILES: COc1cc(OC)nc(NC(=O)NS(=O)(=O)c2ncccc2C2=NOCCO2)n1 ; [Na+]]

and the compound of formula (IV) (=A1-88), i.e. the sodium salt of compound (A1-82)

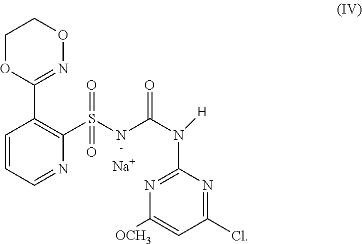 [SMILES: COc1cc(Cl)nc(NC(=O)NS(=O)(=O)c2ncccc2C2=NOCCO2)n1 ; [Na+]]

13. The Beta vulgaris plant, plant part, or plant population according to statement 11, wherein said sulfonylaminocarbonyltriazolinone is selected from one or more of
flucarbazone-sodium [CAS RN 181274-17-9] (=A2-1);
propoxycarbazone-sodium [CAS RN 181274-15-7] (=A2-2); and
thiencarbazone-methyl [CAS RN 317815-83-1] (=A2-3).
14. The Beta vulgaris plant, plant part, or plant population according to statement 11, wherein said triazolopyrimidine is selected from one or more of
cloransulam-methyl [147150-35-4] (=A3-1);
diclosulam [CAS RN 145701-21-9] (=A3-2);
florasulam [CAS RN 145701-23-1] (=A3-3);
flumetsulam [CAS RN 98967-40-9] (=A3-4);
metosulam [CAS RN 139528-85-1] (=A3-5);
penoxsulam [CAS RN 219714-96-2] (=A3-6);
pyroxsulam [CAS RN 422556-08-9] (=A3-7).
15. The Beta vulgaris plant, plant part, or plant population according to statement 11, wherein said sulfonanilide is selected from one or more of
compounds or salts thereof from the group described by the general formula (V):

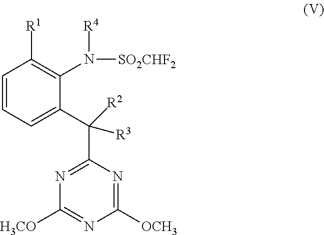

in which
R¹ is halogen, preferably fluorine or chlorine,
R¹ is hydrogen and R<3> is hydroxyl or
R² and R³ together with the carbon atom to which they are attached are a carbonyl group C═O and
R⁴ is hydrogen or methyl;
and more especially compounds of the below given chemical structure (A4-1) to (A4-8)

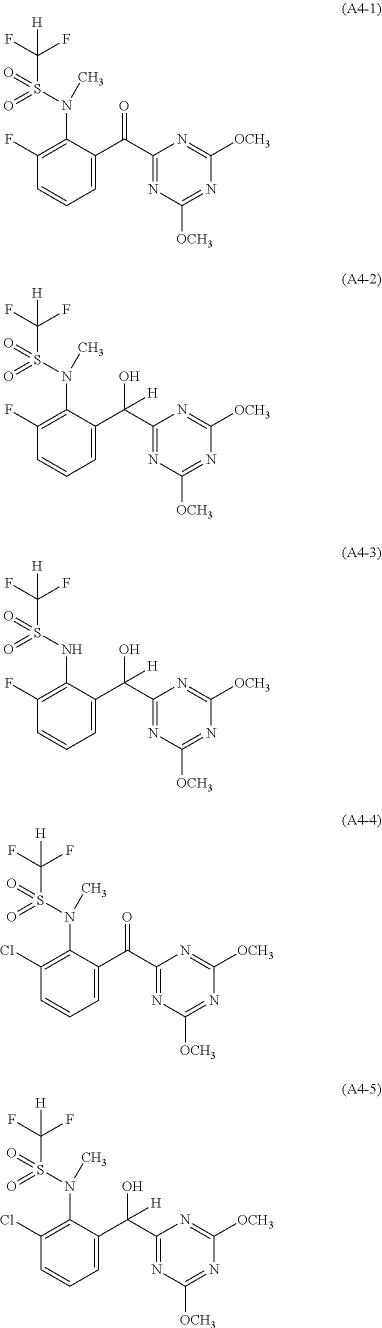

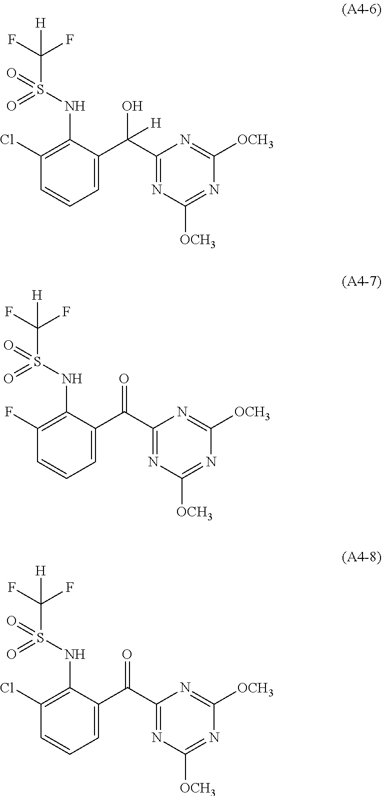 [SMILES: COc1nc(OC)nc(C(O)c2cccc(Cl)c2NS(=O)(=O)C(F)F)n1 ; COc1nc(OC)nc(C(=O)c2cccc(F)c2NS(=O)(=O)C(F)F)n1 ; COc1nc(OC)nc(C(=O)c2cccc(Cl)c2NS(=O)(=O)C(F)F)n1]

16. The Beta vulgaris plant, plant part, or plant population according to statement 11, wherein said imidazolinone is selected from one or more of
imazamethabenzmethyl [CAS RN 81405-85-8] (=B1-1);
imazamox [CAS RN 114311-32-9] (=B1-2);
imazapic [CAS RN 104098-48-8] (=B1-3);
imazapyr [CAS RN 81334-34-1] (=B1-4);
imazaquin [CAS RN 81335-37-7] (=B1-5);
imazethapyr [CAS RN 81335-77-5] (=B1-6);
SYP-298 [CAS RN 557064-77-4] (=B1-7);
SYP-300 [CAS RN 374718-10-2] (=B1-8).
17. The Beta vulgaris plant, plant part, or plant population according to statement 11, wherein said pyrimidinyloxybenzoate is selected from one or more of
bispyribac-sodium [CAS RN 125401-92-5] (=C1-1);
pyribenzoxim [CAS RN 168088-61-7] (=C1-2);
pyriminobac-methyl [CAS RN 136191-64-5] (=C1-3);
pyribambenz-isopropyl [CAS RN 420138-41-6] (=C1-4);
pyribambenz-propyl [CAS RN 420138-40-5] (=C1-5).
18. The Beta vulgaris plant, plant part, or plant population according to statement 11, wherein said pyrimidinylthiobenzoate is selected from one or more of
pyriftalid [CAS RN 135186-78-6] (=C2-1);
pyrithiobac-sodium [CAS RN 123343-16-8] (=C2-2).
19. The Beta vulgaris plant part according to any of statements 1 to 18, wherein said plant part is a root beet, seed, cell, tissue, or organ.
20. The Beta vulgaris plant, plant part, or plant population according to any of statements 1 to 19, comprising a polynucleic acid encoding a mutated endogenous acetolactate synthase (ALS) protein comprising any one or more of
at position 113 an amino acid different than alanine (A);
at position 188 an amino acid different than proline (P);
at position 196 an amino acid different than alanine (A);
at position 372 an amino acid different than arginine (R);
at position 569 an amino acid different than tryptophan (W);
at position 648 an amino acid different than serine (S);
at position 649 an amino acid different than glycine (G).
21. The Beta vulgaris plant, plant part, or plant population according to any of statements 1 to 20, comprising a mutated endogenous allele encoding an ALS protein comprising any one or more of
at position 113 an amino acid different than alanine (A);
at position 188 an amino acid different than proline (P);
at position 196 an amino acid different than alanine (A);
at position 372 an amino acid different than arginine (R);
at position 569 an amino acid different than tryptophan (W);
at position 648 an amino acid different than serine (S);
at position 649 an amino acid different than glycine (G).
22. The Beta vulgaris plant, plant part, or plant population according to any of statements 1 to 20, wherein said ALS protein comprises alanine, glycine, isoleucine, leucine, methionine, phenylalanine, proline, valine or arginine, preferably leucine, at position 569.
23. The Beta vulgaris plant, plant part, or plant population according to any of statements 1 to 22, which is transgenic, gene-edited, or mutagenized.
24. The Beta vulgaris plant, plant part, or plant population according to any of statements 1 to 23, wherein said Beta vulgaris plant or plant part is a sugar beet plant or plant part.
25. A (isolated) polynucleic acid encoding a mutated endogenous acetolactate synthase (ALS) protein as defined in any of statements 1 to 24.
26. The (isolated) polynucleic acid according to statement 25, which is an isolated polynucleic acid.
27. A vector comprising the polynucleic acid according to statement 25 or 26.
28. The (isolated) polynucleic acid or vector according to any of statements 25 to 27, wherein said wherein said polynucleic acid comprises one or more regulatory sequence operably linked to the sequence encoding the ALS protein.
29. A host cell comprising the polynucleic acid or vector according to any of statements 25 to 28.
30. The host cell according to statement 29, which is a plant host cell, preferably a Beta vulgaris host cell.
31. A (isolated) polynucleic acid specifically hybridizing with the polynucleic acid according to any of statements 25 to 28, the complement thereof, or the reverse complement thereof.
32. The (isolated) polynucleic acid according to statement 31, having a length ranging from 10 to 200 nucleotides.
33. The (isolated) polynucleic acid according to statement 31 or 32, which is a primer or a probe.
34. The (isolated) polynucleic acid according to any of statements 31 to 33, which is a KASP primer.
35. The (isolated) polynucleic acid according to any of statements 31 to 34, comprising at least the 10 most 3′ nucleotides of SEQ ID NO: 7, preferably at least the 15 most 3′ nucleotides, the complement thereof, or the reverse complement thereof.
36. The (isolated) polynucleic acid according to any of statements 31 to 35, comprising or consisting of SEQ ID NO: 7, the complement thereof, or the reverse complement thereof.
37. Use of a polynucleic acid according to any of statements 31 to 36 for identifying a Beta vulgaris plant.
38. Use of a polynucleic acid according to any of statements 31 to 36 for identifying a Beta vulgaris plant according to any of statements 1 to 24.
39. Use of a polynucleic acid according to any of statements 31 to 36 for identifying a Beta vulgaris plant which is tolerant to one or more ALS inhibitor herbicide.
40. Use according to statement 39, wherein said ALS inhibitor herbicide is one or more ALS inhibitor herbicide as defined in any of statements 11 to 18.
41. Use of the polynucleic acid, vector, or host cell according to any of statements 25 to 30 for generating a Beta vulgaris plant.
42. Use of the polynucleic acid, vector, or host cell according to any of statements 25 to 30 for generating a Beta vulgaris plant according to any of statements 1 to 24.
43. Use of the polynucleic acid, vector, or host cell according to any of statements 25 to 30 for generating a Beta vulgaris plant which is tolerant to one or more ALS inhibitor herbicide.
44. Use according to statement 40, wherein said ALS inhibitor herbicide is one or more ALS inhibitor herbicide as defined in any of statements 11 to 18.
45. A method for identifying a Beta vulgaris plant or plant part, comprising screening for the presence in an ALS protein of an amino acid at position 371 which is different than aspartic acid (D), or screening for the presence of a codon encoding an amino acid in an ALS protein at position 371 which is different than aspartic acid (D).
46. The method according to statement 45, further comprising selecting the Beta vulgaris plant or plant part comprising an ALS protein comprising at position 371 an amino acid which is different than aspartic acid (D) or comprising a polynucleic acid encoding an ALS protein comprising at position 371 an amino acid different than aspartic acid (D).
47. The method according to statement 45 or 46, comprising screening for the presence of an ALS protein having a sequence which is at least 80% identical to SEQ ID NO: 3, preferably at least 90%, more preferably at least 95%, such as at least 98%, or screening for the presence of a polynucleic acid encoding an ALS protein having a sequence which is at least 80% identical to SEQ ID NO: 3, preferably at least 90%, more preferably at least 95%, such as at least 98%.
48. The method according to any of statements 45 to 47, wherein said plant or plant part expresses or is capable of expressing said ALS protein.
49. The method according to any of statements 45 to 48, wherein said ALS protein has ALS activity.
50. The method according to any of statements 45 to 49, wherein said ALS protein comprises glutamate (E) at position 371.
51. The method according to any of statements 45 to 50, wherein said ALS protein has a sequence of SEQ ID NO: 3.
52. The method according to any of statements 45 to 51, wherein said ALS is heterozygous.
53. The method according to any of statements 45 to 51, wherein said ALS is homozygous.
54. The method according to any of statements 45 to 53, wherein said plant or plant part is tolerant to one or more ALS inhibitor herbicide.
55. The method according to any of statements 45 to 54, wherein said ALS inhibitor herbicide is one or more ALS inhibitor herbicide as defined in any of statements 11 to 18.
56. A method for generating a Beta vulgaris plant or plant part, comprising mutating in the genome of a plant or plant part an endogenous ALS allele resulting in an ALS allele encoding an ALS protein comprising at position 371 an amino acid different than aspartic acid (D).
57. The method according to statement 55, comprising the steps of:
a) mutagenizing Beta vulgaris cells or tissue with at least 0.5% EMS or at least 0.3% ENU,
b) producing stecks from the mutagenized cells or tissue (M0),
c) replanting stecks for producing a population of seeds (M1),
d) producing seeds (M2) from plants grown from M1 seeds,
e) sowing M2 seeds and applying an ALS inhibitor herbicide,
f) optionally, replanting surviving plants in pots and applying an ALS inhibitor herbicide to the growing plants, and
g) selecting surviving plants without herbicide damages and/or selecting plants comprising an ALS allele encoding an ALS protein comprising at position 371 an amino acid different than aspartic acid (D).
58. A method for generating a Beta vulgaris plant or plant part, comprising introducing in the genome (and expressing) of a plant or plant part a polynucleic acid encoding a mutated endogenous ALS protein comprising at position 371 an amino acid different than aspartic acid (D).
59. The method according to statement 58, wherein said polynucleic acid comprises one or more regulatory sequence operably linked to the sequence encoding the ALS protein.
60. The method according to statement 58 or 59, wherein introducing in the genome comprises transgenesis, gene-editing, or mutagenesis.
61. The method according to any of statements 58 to 60, comprising transforming a plant or plant part, preferably a plant cell, more preferably a protoplast, with a polynucleic acid or vector according to any of statements 25 to 29, and optionally regenerating a plant from said plant cell, preferably protoplast.
62. The method according to statement 58 or 59, wherein introducing in the genome comprises introgression.
63. A Beta vulgaris plant or plant part (directly) obtained by or obtainable by the method according to any of statements 56 to 62, or the progeny thereof.
64. The plant part according to statement 63, wherein said plant part is a root beet, seed, cell, tissue, or organ.
65. A method for controlling unwanted vegetation in Beta vulgaris growing areas or for increasing the yield in Beta vulgaris growing areas, comprising the steps of:
a) planting Beta vulgaris plants or sowing Beta vulgaris seeds according to any of statements 1 to 24,
b) applying one or more ALS inhibitor herbicide to the growing plants, preferably at a dosage sufficient for inhibiting the growth of the unwanted vegetation, more preferably at a dosage sufficient for killing the unwanted vegetation, and
c) optionally, repeating step b) during the growing season.
66. The method according to statement 64, wherein step b) is performed before pollination of unwanted vegetation, preferably during pre-flowering stage or latest at the time when flowers open.
67. The method according to statement 65 or 66, wherein said yield is root beet yield.
68. Use of one or more ALS inhibitor herbicide for controlling unwanted vegetation in Beta vulgaris growing areas, in which the Beta vulgaris plants are according to any of statements 1 to 24.
69. The method or use according to any of statements 65 to 68, wherein said unwanted vegetation is or comprises bolters, weed beets, or annual beets.
70. The method or use according to any of statements 65 to 69, wherein said one or more ALS inhibitor herbicide is one or more ALS inhibitor herbicide as defined in any of statements 11 to 18.
71. A method for producing Beta vulgaris root beets, comprising the steps of:
a) conducting the method of any of statements 65 to 67, 69, or 70, and
b) harvesting Beta vulgaris root beets, preferably by the end of the growing season.
72. Use of a Beta vulgaris plant, plant part, or plant population according to any of statements 1 to 24 in a method for sugar production, anaerobic digestion, or fermentation.
73. Use of a Beta vulgaris plant, plant part, or plant population according to any of statements 1 to 24 in a method for biogas or biofuel production.
74. A Beta vulgaris plant, plant part, or plant population comprising:
a) a polynucleic acid comprising a sequence as set forth in SEQ ID NO: 1;
b) a polynucleic acid comprising a sequence as set forth in SEQ ID NO: 2;
c) a polynucleic acid encoding an ALS protein having a cDNA sequence as set forth in SEQ ID NO: 2;
d) a polynucleic acid encoding an ALS protein having a sequence as set forth in SEQ ID NO: 3.
75. The Beta vulgaris plant, plant part, or plant population according to statement 75, comprising:
a) a polynucleic acid comprising a sequence as set forth in SEQ ID NO: 10;
b) a polynucleic acid comprising a sequence as set forth in SEQ ID NO: 11;
c) a polynucleic acid encoding an ALS protein having a cDNA sequence as set forth in SEQ ID NO: 11;
d) a polynucleic acid encoding an ALS protein having a sequence as set forth in SEQ ID NO: 12.
76. A (isolated) polynucleic acid comprising a sequence as set forth in SEQ ID NO: 7.

In an aspect, the invention relates to a Beta vulgaris plant, plant part, or plant population comprising, expressing, or capable of expressing a mutated endogenous acetolactate synthase (ALS) protein or a polynucleic acid encoding a mutated endogenous acetolactate synthase (ALS) protein comprising at position 371 an amino acid different than aspartic acid (D). in an embodiment, the Beta vulgaris plant, plant part, or plant population comprises a mutated endogenous allele encoding an ALS protein comprising at position 371 an amino acid different than aspartic acid (D).

In an aspect, the invention relates to a Beta vulgaris plant, plant part, or plant population comprising a mutated endogenous allele encoding an ALS protein comprising at position 371 an amino acid different than aspartic acid (D).

A plant of the species Beta vulgaris is, in particular, a plant of the subspecies Beta vulgaris subsp. vulgaris. For example, numbering among these are Beta vulgaris subsp. vulgaris var. altissima (sugar beet in a narrower sense), Beta vulgaris ssp. vulgaris var. favescens (chard), Beta vulgaris ssp. vulgaris var. cicla (spinach beet), Beta vulgaris ssp. vulgaris var. conditiva (beetroot/red beet/garden beet), Beta vulgaris ssp. vulgaris var. crassa/alba (fodder beet). In a preferred embodiment, Beta vulgaris as referred to herein according to the invention is Beta vulgaris subsp. vulgaris, more preferably Beta vulgaris subsp. vulgaris var. altissima (i.e. sugar beet).

As used herein, ALS (acetolactate synthase; also known as AHAS (acetohydroxyacid synthase); EC 2.2.1.6; formerly EC 4.1.3.18)) is involved in the conversion of two pyruvate molecules to an acetolactate molecule and carbon dioxide. The reaction uses thyamine pyrophosphate in order to link the two pyruvate molecules. The resulting product of this reaction, acetolactate, eventually becomes valine, leucine and isoleucine (Singh (1999) “Biosynthesis of valine, leucine and isoleucine”, in Plant Amino Acids, Singh, B. K., ed., Marcel Dekker Inc. New York, New York, pp. 227-247). Inhibitors of the ALS interrupt the biosynthesis of valine, leucine and isoleucine in plants. The consequence is an immediate depletion of the respective amino acid pools causing a stop of protein biosynthesis leading to a cessation of plant growth and eventually the plant dies, or—at least—is damaged.

In certain embodiments, the wild type Beta vulgaris ALS has an amino acid sequence as provided in SEQ ID NO: 6. In certain embodiments, the wild type or native Beta vulgaris ALS has an amino acid sequence having at least 80%, preferably at least 90%, more preferably at least 95%, most preferably at least 98%, such as at least 99% sequence identity, preferably over the entire length, to the sequence of SEQ ID NO: 6, and preferably has ALS activity, with the proviso that amino acid residue at position 371 is aspartic acid (D). It will be understood that aspactic acid as used herein may be used interchangeably with aspartate. In certain embodiments, the wild type Beta vulgaris ALS has an amino acid sequence as provided in NCBI reference sequence XP_010695365.1. In certain embodiments, the wild type or native Beta vulgaris ALS has an amino acid sequence having at least 80%, preferably at least 90%, more preferably at least 95%, most preferably at least 98%, such as at least 99% sequence identity, preferably over the entire length, to the sequence of NCBI reference sequence XP_010695365.1, and preferably has ALS activity, with the proviso that amino acid residue at position 371 is aspartic acid (D).

In certain embodiments, the mutated Beta vulgaris ALS according to the invention has an amino acid sequence as provided in SEQ ID NO: 3. In certain embodiments, the mutated Beta vulgaris ALS has an amino acid sequence having at least 80%, preferably at least 90%, more preferably at least 95%, most preferably at least 98%, such as at least 99% sequence identity, preferably over the entire length, to the sequence of SEQ ID NO: 3, and preferably has ALS activity, with the proviso that amino acid residue at position 371 is not aspartic acid (D). In certain embodiments, the mutated Beta vulgaris ALS has an amino acid sequence as provided in NCBI reference sequence XP_010695365.1. In certain embodiments, the mutated Beta vulgaris ALS has an amino acid sequence having at least 80%, preferably at least 90%, more preferably at least 95%, most preferably at least 98%, such as at least 99% sequence identity, preferably over the entire length, to the sequence of NCBI reference sequence XP_010695365.1, and preferably has ALS activity, with the proviso that amino acid residue at position 371 is not aspartic acid (D).

In certain embodiments, the wild type Beta vulgaris ALS gene has a sequence encoding an amino acid sequence as provided in SEQ ID NO: 6. In certain embodiments, the wild type or native Beta vulgaris ALS gene has a sequence encoding an amino acid sequence having at least 80%, preferably at least 90%, more preferably at least 95%, most preferably at least 98%, such as at least 99% sequence identity, preferably over the entire length, to the sequence of SEQ ID NO: 6, and preferably has ALS synthase activity, with the proviso that amino acid residue at position 371 is aspartic acid (D). In certain embodiments, the wild type Beta vulgaris ALS gene has a sequence encoding an amino acid sequence as provided in NCBI reference sequence XP_010695365.1. In certain embodiments, the wild type or native Beta vulgaris ALS gene has a sequence encoding an amino acid sequence having at least 80%, preferably at least 90%, more preferably at least 95%, most preferably at least 98%, such as at least 99% sequence identity, preferably over the entire length, to the sequence of NCBI reference sequence XP_010695365.1, and preferably has ALS synthase activity, with the proviso that amino acid residue at position 371 is aspartic acid (D).

In certain embodiments, the mutated Beta vulgaris ALS gene according to the invention has a sequence encoding an amino acid sequence as provided in SEQ ID NO: 3. In certain embodiments, the mutated Beta vulgaris ALS gene has a sequence encoding an amino acid sequence having at least 80%, preferably at least 90%, more preferably at least 95%, most preferably at least 98%, such as at least 99% sequence identity, preferably over the entire length, to the sequence of SEQ ID NO: 3, and preferably has ALS synthase activity, with the proviso that amino acid residue at position 371 is not aspartic acid (D). In certain embodiments, the mutated Beta vulgaris ALS gene has a sequence encoding an amino acid sequence as provided in NCBI reference sequence XP_010695365.1. In certain embodiments, the mutated Beta vulgaris ALS gene has a sequence encoding an amino acid sequence having at least 80%, preferably at least 90%, more preferably at least 95%, most preferably at least 98%, such as at least 99% sequence identity, preferably over the entire length, to the sequence of NCBI reference sequence XP_010695365.1, and preferably has ALS synthase activity, with the proviso that amino acid residue at position 371 is not aspartic acid (D).

In certain embodiments, the wild type Beta vulgaris ALS gene has a nucleotide sequence as provided in SEQ ID NO: 4. In certain embodiments, the wild type or native Beta vulgaris ALS gene has a nucleotide sequence having at least 80%, preferably at least 90%, more preferably at least 95%, most preferably at least 98%, such as at least 99% sequence identity, preferably over the entire length, to the sequence of SEQ ID NO: 4, and preferably has ALS activity, with the proviso that the codon corresponding to amino acid residue at position 371 encodes aspartic acid (D).

In certain embodiments, the mutated Beta vulgaris ALS gene according to the invention has a nucleotide sequence as provided in SEQ ID NO: 1. In certain embodiments, the mutated Beta vulgaris ALS gene has a nucleotide sequence having at least 80%, preferably at least 90%, more preferably at least 95%, most preferably at least 98%, such as at least 99% sequence identity, preferably over the entire length, to the sequence of SEQ ID NO: 1, and preferably has ALS activity, with the proviso that the codon corresponding to amino acid residue at position 371 does not encode aspartic acid (D).

In certain embodiments, the wild type Beta vulgaris ALS coding sequence (cDNA) has a nucleotide sequence as provided in SEQ ID NO: 5. In certain embodiments, the wild type or native Beta vulgaris ALS coding sequence has a nucleotide sequence having at least 80%, preferably at least 90%, more preferably at least 95%, most preferably at least 98%, such as at least 99% sequence identity, preferably over the entire length, to the sequence of SEQ ID NO: 5, and preferably has ALS activity, with the proviso that the codon corresponding to amino acid residue at position 371 encodes aspartic acid (D).

In certain embodiments, the mutated Beta vulgaris ALS coding sequence (cDNA) according to the invention has a nucleotide sequence as provided in SEQ ID NO: 2. In certain embodiments, the mutated Beta vulgaris ALS coding sequence has a nucleotide sequence having at least 80%, preferably at least 90%, more preferably at least 95%, most preferably at least 98%, such as at least 99% sequence identity, preferably over the entire length, to the sequence of SEQ ID NO: 2, and preferably has ALS activity, with the proviso that the codon corresponding to amino acid residue at position 371 does not encode aspartic acid (D).

Preferably, as used herein, where amino acid residue positions are referred to for the ALS, the numbering corresponds to the amino acid positions in SEQ ID NO: 6. SEQ ID NO: 3 corresponds to the sequence of SEQ ID NO: 6 having the D371E mutation.

The term “position” when used in accordance with the present invention means the position of either an amino acid within an amino acid sequence depicted herein or the position of a nucleotide within a nucleotide sequence depicted herein. The term “corresponding” as used herein also includes that a position is not only determined by the number of the preceding nucleotides/amino acids.

The position of a given nucleotide in accordance with the present invention which may be substituted may vary due to deletions or additional nucleotides elsewhere in the ALS 5′-untranslated region (UTR) including the promoter and/or any other regulatory sequences or gene (including exons and introns). Similarly, the position of a given amino acid in accordance with the present invention which may be substituted may vary due to deletion or addition of amino acids elsewhere in the ALS polypeptide.

Thus, under a “corresponding position” in accordance with the present invention it is to be understood that nucleotides/amino acids may differ in the indicated number but may still have similar neighbouring nucleotides/amino acids. Said nucleotides/amino acids which may be exchanged, deleted or added are also comprised by the term “corresponding position”.

In order to determine whether a nucleotide residue or amino acid residue in a given ALS nucleotide/amino acid sequence corresponds to a certain position such as in the nucleotide sequence of SEQ ID NOs: 1, 2, 4, or 5, or the amino acid sequence of SEQ ID NO: 3 or 6, the skilled person can use means and methods well-known in the art, e.g., alignments, either manually or by using computer programs such as BLAST (Altschul et al. (1990), Journal of Molecular Biology, 215, 403-410), which stands for Basic Local Alignment Search Tool or ClustalW (Thompson et al. (1994), Nucleic Acid Res., 22, 4673-4680) or any other suitable program which is suitable to generate sequence alignments.

In view of the difference between the B. vulgaris wild-type ALS gene and the ALS gene comprised by a B. vulgaris plant of the present invention, the ALS gene (or polynucleotide or nucleotide sequence) comprised by a B. vulgaris plant of the present invention may also be regarded as a “mutant ALS gene”, “mutant ALS allele”, “mutant ALS polynucleotide” or the like. Thus, throughout the specification, the terms “mutant allele”, “mutant ALS allele”, “mutant ALS gene” or “mutant ALS polynucleotide” are used interchangeably.

In contrast, unless indicated otherwise, the terms “wild-type allele,” “wild-type ALS allele”, “wild-type ALS gene” or “wild-type ALS polynucleotide” refer to a nucleotide sequence that encodes an ALS protein that lacks the D371 substitution. Such a “wild-type allele”, “wild-type ALS allele”, “wild-type ALS gene” or “wild-type ALS polynucleotide” may, or may not, comprise mutations, other than the mutation that causes the D371 substitution. Also naturally occurring polymorphisms (other than at position 371) in ALS can be considered to be covered by the term “wild-type”.

As used herein, the term “ALS activity” refers to the enzymatic activity of the ALS protein. The term “having ALS activity” in the context of variant ALS as described above (such as the ALS proteins having a certain percentage sequence identity to recited SEQ ID NOs) in certain preferred embodiments refers to an ALS of which the enzymatic activity is unaffected or substantially unaffected compared to wild type or native ALS (such as an ALS having a sequence of a recited SEQ ID NO). In certain embodiments, the enzymatic activity is at least 50% of the wild type ALS activity, preferably at least 60%, more preferably at least 70%, even more preferably at least 80%, most preferably at least 90%, such as at least 95%. Enzymatic activity can be measured by means known in the art, such as determination of conversion of pyruvate into acetolactate.

In certain embodiments, the Beta vulgaris plants according to the invention have a (mutated) ALS of which the enzymatic activity is unaffected or substantially unaffected by one or more ALS inhibitor herbicides. In certain embodiments, the Beta vulgaris plants according to the invention have an ALS of which the enzymatic activity is at most 50% less in the presence of one or more ALS inhibitor herbicides compared to the absence of such herbicides, preferably at most 40% less, more, preferably at most 30% less, even more preferably at most 20 less, most preferably at most 10% less, such as at most 5% less. Enzymatic activity can be measured by means known in the art. Enzymatic activity is preferably determined in the presence of an ALS inhibitor herbicide at a relevant applicable herbicidal dose, such as a dose corresponding to a field application as recommended.

In certain embodiments, the mutated ALS according to the invention, i.e. having an amino acid at position 371 which is different from aspartic acid, comprises at position 371 a similar amino acid. In certain embodiments, the mutated ALS according to the invention, i.e. having an amino acid at position 371 which is different from aspartic acid, comprises at position 371 a conservative substitution. In certain embodiments, the mutated ALS according to the invention, i.e. having an amino acid at position 371 which is different from aspartic acid, comprises at position 371 a non-similar amino acid. In certain embodiments, the mutated ALS according to the invention, i.e. having an amino acid at position 371 which is different from aspartic acid, comprises at position 371 a non-conservative substitution. In certain embodiments, the mutated ALS according to the invention, i.e. having an amino acid at position 371 which is different from aspartic acid, comprises at position 371 a polar amino acid. In certain embodiments, the mutated ALS according to the invention, i.e. having an amino acid at position 371 which is different from aspartic acid, comprises at position 371 an amino acid selected from glutamine—Gln—Q, asparagine—Asn—N, histidine—His—H, serine—Ser—S, threonine—Thr—T, tyrosine—Tyr—Y, cysteine—Cys—C. In certain embodiments, the mutated ALS according to the invention, i.e. having an amino acid at position 371 which is different from aspartic acid, comprises at position 371 an acidic amino acid. In certain embodiments, the mutated ALS according to the invention, i.e. having an amino acid at position 371 which is different from aspartic acid, comprises at position 371 an acidic polar amino acid. In certain embodiments, the mutated ALS according to the invention, i.e. having an amino acid at position 371 which is different from aspartic acid, comprises at position 371 a glutamic acid (glutamate, E). In certain embodiments, the mutated ALS according to the invention, i.e. having an amino acid at position 371 which is different from aspartic acid, comprises at position 371 a non-polar amino acid.

As used herein, the term “capable of expressing” means that a protein can be expressed in a plant or plant part. As such, it requires that a gene sequence of a protein or a coding sequence of a protein is present in the plant or plant part, preferably in the genome of the plant or plant part. Appropriate regulatory sequences should also be present to ensure transcription. Accordingly, the ALS encoding polynucleotide should be operably linked to one or more regulatory sequence, such as a promoter. However, it (can be but) is not necessary that transcription is ubiquitous (constitutive). Transcription may be cell-, tissue-, or organ-specific. Transcription may alternatively or in addition be developmentally-specific (i.e. only at certain developmental stages is the protein expressed). Transcription may alternatively or additionally be conditional or inducible. Suitable promoters for each of these instances are known in the art. In certain preferred embodiments, the mutated ALS gene according to the invention is located at its native (endogenous) position (locus) in the genome, and hence is under control of its native (endogenous) promoter.

As used herein, the term “operatively linked” or “operably linked” means connected in a common nucleic acid molecule in such a manner that the connected elements are positioned and oriented relative to one another such that a transcription of the nucleic acid molecule may occur. A DNA which is operatively linked with a promoter is under the transcriptional control of this promoter.

As used herein unless clearly indicated otherwise, the term “plant” intended to mean a plant at any developmental stage.

It is preferred that the Beta vulgaris plant of the present invention is orthoploid or anorthoploid. An orthoploid plant may preferably be haploid, diploid, tetraploid, hexaploid, octaploid, decaploid or dodecaploid, while an anorthoploid plant may preferably be triploid or pentaploid. In certain preferred embodiments, the Beta vulgaris plant according to the invention is diploid.

The term “plant” according to the present invention includes whole plants or parts of such a whole plant. Whole plants preferably are seed plants, or a crop. “Parts of a plant” are e.g. shoot vegetative organs/structures, e.g., leaves, stems and tubers; roots, flowers and floral organs/structures, e.g. bracts, sepals, petals, stamens, carpels, anthers and ovules; seed, including embryo, endosperm, and seed coat; fruit and the mature ovary; plant tissue, e.g. vascular tissue, ground tissue, and the like; and cells, e.g. guard cells, egg cells, pollen, trichomes and the like; and progeny of the same. Parts of plants may be attached to or separate from a whole intact plant. Such parts of a plant include, but are not limited to, organs, tissues, and cells of a plant, and preferably seeds. A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant. “Plant cell culture” means cultures of plant units such as, for example, protoplasts, cell culture cells, cells in plant tissues, pollen, pollen tubes, ovules, embryo sacs, zygotes and embryos at various stages of development. “Plant material” refers to leaves, stems, roots, flowers or flower parts, fruits, pollen, egg cells, zygotes, seeds, cuttings, cell or tissue cultures, or any other part or product of a plant. This also includes callus or callus tissue as well as extracts (such as extracts from taproots) or samples. A “plant organ” is a distinct and visibly structured and differentiated part of a plant such as a root, stem, leaf, flower bud, or embryo. “Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue. In certain preferred embodiments, the plant parts or plant organs as referred to herein are root beet (or rootbeet) or seed. The term root beet (or beetroot) refers to the taproot or hypocotyl or the beet which has been transformed into a fleshy storage organ. In certain embodiments, the plant part as used herein is a protoplast.

As used herein, the term “plant population” may be used interchangeably with population of plants. A plant population preferably comprises a multitude of individual plants, such as preferably at least 10, such as 20, 30, 40, 50, 60, 70, 80, or 90, more preferably at least 100, such as 200, 300, 400, 500, 600, 700, 800, or 900, even more preferably at least 1000, such as at least 10000 or at least 100000.

The term “sequence” when used herein relates to nucleotide sequence(s), polynucleotide(s), nucleic acid sequence(s), nucleic acid(s), nucleic acid molecule, peptides, polypeptides and proteins, depending on the context in which the term “sequence” is used.

The terms “nucleotide sequence(s)”, “polynucleotide(s)”, “nucleic acid sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length. Nucleic acid sequences include DNA, cDNA, genomic DNA, RNA, synthetic forms and mixed polymers, both sense and antisense strands, or may contain non-natural or derivatized nucleotide bases, as will be readily appreciated by those skilled in the art.

When used herein, the term “polypeptide” or “protein” (both terms are used interchangeably herein) means a peptide, a protein, or a polypeptide which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention as well as other than the 20 gene-encoded amino acids, such as selenocysteine. Peptides, oligopeptides and proteins may be termed polypeptides. The term polypeptide also refers to, and does not exclude, modifications of the polypeptide, e.g., glycosylation, acetylation, phosphorylation and the like. Such modifications are well described in basic texts and in more detailed monographs, as well as in the research literature.

Amino acid substitutions encompass amino acid alterations in which an amino acid is replaced with a different naturally-occurring amino acid residue. Such substitutions may be classified as “conservative”, in which an amino acid residue contained in the wild-type protein is replaced with another naturally-occurring amino acid of similar character, for example Gly< >Ala, Val< >Ile< >Leu, Asp< >Glu, Lys< >Arg, Asn< >Gln or Phe< >Trp< >Tyr. Substitutions encompassed by the present invention may also be “non-conservative”, in which an amino acid residue which is present in the wild-type protein is substituted with an amino acid with different properties, such as a naturally-occurring amino acid from a different group (e.g. substituting a charged or hydrophobic amino acid with alanine. “Similar amino acids”, as used herein, refers to amino acids that have similar amino acid side chains, i.e. amino acids that have polar, non-polar or practically neutral side chains. “Non-similar amino acids”, as used herein, refers to amino acids that have different amino acid side chains, for example an amino acid with a polar side chain is non-similar to an amino acid with a non-polar side chain. Polar side chains usually tend to be present on the surface of a protein where they can interact with the aqueous environment found in cells (“hydrophilic” amino acids). On the other hand, “non-polar” amino acids tend to reside within the centre of the protein where they can interact with similar non-polar neighbours (“hydrophobic” amino acids”). Examples of amino acids that have polar side chains are arginine, asparagine, aspartic acid, cysteine, glutamine, glutamate, histidine, lysine, serine, and threonine (all hydrophilic, except for cysteine which is hydrophobic). Examples of amino acids that have non-polar side chains are alanine, glycine, isoleucine, leucine, methionine, phenylalanine, proline, and tryptophan (all hydrophobic, except for glycine which is neutral).

The term “gene” when used herein refers to a polymeric form of nucleotides of any length, either ribonucleotides or desoxyribonucleotides. The term includes double- and single-stranded DNA and RNA. It also includes known types of modifications, for example, methylation, “caps”, substitutions of one or more of the naturally occurring nucleotides with an analog. Preferably, a gene comprises a coding sequence encoding the herein defined polypeptide. A “coding sequence” is a nucleotide sequence which is transcribed into mRNA and/or translated into a polypeptide when placed or being under the control of appropriate regulatory sequences. The boundaries of the coding sequence are determined by a translation start codon at the 5-terminus and a translation stop codon at the 3′-terminus. A coding sequence can include, but is not limited to mRNA, cDNA, recombinant nucleic acid sequences or genomic DNA, while introns may be present as well under certain circumstances.

A used herein, the term “endogenous” refers to a gene or allele which is present in its natural genomic location. The term “endogenous” can be used interchangeably with “native”. This does not however exclude the presence of one or more nucleic acid differences with the wild-type allele. In particular embodiments, the difference with a wild-type allele can be limited to less than 9 preferably less than 6, more particularly less than 3 nucleotide differences. More particularly, the difference with the wildtype sequence can be in only one nucleotide. Preferably, the endogenous allele encodes a modified protein having less than 9, preferably less than 6, more particularly less than 3 and even more preferably only one amino acid difference with the wild-type protein.

As used herein, the term “homozygote” refers to an individual cell or plant having the same alleles at one or more or all loci. When the term is used with reference to a specific locus or gene, it means at least that locus or gene has the same alleles. As used herein, the term “homozygous” means a genetic condition existing when identical alleles reside at corresponding loci on homologous chromosomes. As used herein, the term “heterozygote” refers to an individual cell or plant having different alleles at one or more or all loci. When the term is used with reference to a specific locus or gene, it means at least that locus or gene has different alleles. As used herein, the term “heterozygous” means a genetic condition existing when different alleles reside at corresponding loci on homologous chromosomes.

As used herein, an “allele” refers to alternative forms of various genetic units associated with different forms of a gene or of any kind of identifiable genetic element, which are alternative in inheritance because they are situated at the same locus in homologous chromosomes. In a diploid cell or organism, the two alleles of a given gene (or marker) typically occupy corresponding loci on a pair of homologous chromosomes.

The term “locus” (loci plural) means a specific place or places or a site on a chromosome where for example a QTL, a gene or genetic marker is found, such as a (mutant) ALS encoding sequence of the invention.

As used herein, the term “sequence identity” refers to the degree of identity between any given nucleic acid sequence and a target nucleic acid sequence. Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (Bl2seq) program from the stand-alone version of BLASTZ containing BLASTN and BLASTP. This stand-alone version of BLASTZ can be obtained from Fish & Richardson's web site (World Wide Web at fr.com/blast) or the U.S. government's National Center for Biotechnology Information web site (World Wide Web at ncbi.nlm.nih.gov). Instructions explaining how to use the B12seq program can be found in the readme file accompanying BLASTZ. B12seq performs a comparison between two sequences using either the BLASTN or BLASTP algorithm.

BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq I .txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. The following command will generate an output file containing a comparison between two sequences: C:\Bl2seq -i c:\seql .txt -j c:\seq2.txt -p blastn -o c:\output.txt -q-1 -r 2. If the target sequence shares homology with any portion of the identified sequence, then the designated output file will present those regions of homology as aligned sequences. If the target sequence does not share homology with any portion of the identified sequence, then the designated output file will not present aligned sequences. Once aligned, a length is determined by counting the number of consecutive nucleotides from the target sequence presented in alignment with the sequence from the identified sequence starting with any matched position and ending with any other matched position. A matched position is any position where an identical nucleotide is presented in both the target and identified sequences. Gaps presented in the target sequence are not counted since gaps are not nucleotides. Likewise, gaps presented in the identified sequence are not counted since target sequence nucleotides are counted, not nucleotides from the identified sequence. The percent identity over a particular length is determined by counting the number of matched positions over that length and dividing that number by the length followed by multiplying the resulting value by 100. For example, if (i) a 500-base nucleic acid target sequence is compared to a subject nucleic acid sequence, (ii) the B12seq program presents 200 bases from the target sequence aligned with a region of the subject sequence where the first and last bases of that 200-base region are matches, and (iii) the number of matches over those 200 aligned bases is 180, then the 500-base nucleic acid target sequence contains a length of 200 and a sequence identity over that length of 90% (i.e., 180/200×100=90). It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

An “isolated nucleic acid” is understood to be a nucleic acid isolated from its natural or original environment. The term also includes a synthetic manufactured nucleic acid. Accordingly, an “isolated nucleic acid sequence” or “isolated DNA” refers to a nucleic acid sequence which is no longer in the natural environment from which it was isolated, e.g. the nucleic acid sequence in a bacterial host cell or in the plant nuclear or plastid genome. When referring to a “sequence” herein, it is understood that the molecule having such a sequence is referred to, e.g. the nucleic acid molecule. A “host cell” or a “recombinant host cell” or “transformed cell” are terms referring to a new individual cell (or organism) arising as a result of at least one nucleic acid molecule, having been introduced into said cell. The host cell is preferably a plant cell or a bacterial cell. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

When reference is made to a nucleic acid sequence (e.g. DNA or genomic DNA) having “substantial sequence identity to” a reference sequence or having a sequence identity of at least 80%>, e.g. at least 85%, 90%, 95%, 98%> or 99%> nucleic acid sequence identity to a reference sequence, in one embodiment said nucleotide sequence is considered substantially identical to the given nucleotide sequence and can be identified using stringent hybridisation conditions. In another embodiment, the nucleic acid sequence comprises one or more mutations compared to the given nucleotide sequence but still can be identified using stringent hybridisation conditions. “Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

The term “hybridizing” or “hybridization” means a process in which a single-stranded nucleic acid molecule attaches itself to a complementary nucleic acid strand, i.e. agrees with this base pairing. Standard procedures for hybridization are described, for example, in Sambrook et al. (Molecular Cloning. A Laboratory Manual, Cold Spring Harbor Laboratory Press, 3rd edition 2001). Preferably this will be understood to mean an at least 50%, more preferably at least 55%, 60%, 65%, 70%, 75%, 80% or 85%, more preferably 90%, 91%, 92%, 93%, 94%, 95%, 96%, 97%, 98% or 99% of the bases of the nucleic acid strand form base pairs with the complementary nucleic acid strand. The possibility of such binding depends on the stringency of the hybridization conditions. The term “stringency” refers to hybridization conditions. High stringency is if base pairing is more difficult, low stringency, when a base-pairing is facilitated. The stringency of hybridization conditions depends for example on the salt concentration or ionic strength and temperature. Generally, the stringency can be increased by increasing the temperature and/or decreasing salinity. “Stringent hybridization conditions” are defined as conditions in which hybridization occurs predominantly only between homologous nucleic acid molecules. The term “hybridization conditions” refers not only to the actual binding of the nucleic acids at the prevailing conditions, but also in the subsequent washing steps prevailing conditions. Stringent hybridization conditions are, for example, conditions under which predominantly only those nucleic acid molecules having at least 70%, preferably at least 75%, at least 80%, at least 85%, at least 90% or at least 95% sequence identity hybridize. Less stringent hybridization conditions include: hybridization in 4×SSC at 37° C., followed by repeated washing in 1×SSC at room temperature. Stringent hybridization conditions include: hybridization in 4×SSC at 65° C., followed by repeated washing in 0.1×SSC at 65° C. for a total of about 1 hour. In certain embodiments, polynucleotides which hybridize with certain other polynucleotides are said to hybridize under stringent hybridization conditions.

In certain aspects, the invention relates to Beta vulgaris plants or plant parts which tolerate one or more ALS inhibitor, in particular is doses sufficiently high to effect optimal herbicidal activity. In certain embodiments, the Beta vulgaris plants as described herein tolerate one or more ALS inhibitor at a recommended dose for herbicidal activity. Preferably, said dose is a single application dose. It will be understood that if multiple applications are needed during the growing season, the Beta vulgaris plant according to the invention are preferably tolerant to said multiple applications. Preferably, the ALS inhibitor tolerant Beta vulgaris plant according to the invention has no disadvantages with respect to other important agronomic properties such as growth, yield, quality, pathogen resistance, physiological functions, etc.

As used herein, “resistant”, “resistance”, “tolerance” or “tolerant” means that the application of one or more ALS inhibitor herbicide(s), such as those described herein elsewhere, does not show any apparent effect(s) concerning the physiological functions/phytotoxicity when applied to the respective Beta vulgaris plant, especially sugar beet containing an ALS polypeptide comprising a mutation at position 371 and whereas the application of the same amount of the respective ALS inhibitor herbicide(s) on non-tolerant Beta vulgaris plants leads to significant negative effects concerning plant growth, its physiological functions or shows phytotoxic symptoms. Quality and quantity of the observed effects may depend on the chemical composition of the respective ALS inhibitor herbicide(s) applied, dose rate and timing of the application as well growth conditions/stage of the treated plants.

As used herein the terms “increased tolerance” and “increased resistance” relate to any relief from, reduced presentation of, improvement of, or any combination thereof of any symptom (such as damage or loss in biomass) of the application of an ALS inhibitor herbicide. Increased resistance or tolerance as referred to herein may also relate to the ability to which a plant maintains for instance its biomass production (such as harvestable biomass production, such as seed yield) upon or after ALS inhibitor herbicide application. An ALS inhibitor herbicide resistant or tolerant plant, plant cell or plant part may refer herein to a plant, plant cell or plant part, respectively, having increased resistance/tolerance to an ALS inhibitor herbicide compared to a parent plant from which they are derived (and not having ALS protein having at position 371 an amino acid which is different than aspartic acid). Resistance or tolerance may relate herein to a plant's ability to reduce the effect of one or more ALS inhibitor herbicide on its fitness, yield, biomass (production), etc. Methods of determining herbicide resistance/tolerance are known to the person of skill in the art, such as visual scoring of herbicide-induced damage, determination of biomass (yield), etc.

ALS inhibitor tolerance can for instance be determined by visual injury ratings for plant vigour and plant chlorosis based on a scale from 0 (dead plant) to 9 (completely unaffected plant), such as for instance ratings taken on individual plants 2 weeks after glyphosate application. Ratings of 0 to 3 are characteristic of susceptible plants. Ratings of 3 to 7 indicate a low to intermediate level of tolerance, and ratings of 8 or 9 indicate good levels of tolerance. In particular the ratings have the following meaning: 9. Unaffected plant identical to untreated control; 8. Only very small necrosis on the tips of the leaves with less than 5% of the leaf area affected and yellow; 7. Very small necrosis on the tips of the leaves which start to curl; less than 5% of the leaf area are affected and yellow; 6,5,4. Increasing necrosis and leaf curl; leaves are becoming smaller than normal; 3,2. No or very limited leaf growth; all leaves are curled and affected by necrosis; 1. No growth of the plant; up to 5% of the plant stay green; 0. Dead plant. In certain preferred embodiments, the Beta vulgaris plants according to the invention have a rating of at least 3, preferably at least 7, more preferably at least 8, even more preferably 9.

In certain embodiments, the Beta vulgaris plants according to the invention are less sensitive to an ALS inhibitor herbicide, than the corresponding wild type Beta vulgaris plants. In certain embodiments, the Beta vulgaris plant according to the invention are at least 10 times less sensitive, such as 100 times less sensitive, more preferably, 500 times, even more preferably 1000 times and most preferably less than 2000 times. As used herein, the terms “increased tolerance” and “increased resistance” may be used interchangeably with “reduced sensitivity” or “reduced susceptibility”. Accordingly, a plant, plant part, or plant population according to the invention which is more resistant or more tolerant towards one or more ALS inhibitor herbicide is considered less sensitive toward such herbicide. Less sensitive or less susceptible when used herein may be seen as “more tolerant” or “more resistant”. Similarly, “more tolerant” or “more resistant” may, vice versa, be seen as “less sensitive” or “less susceptible”. More sensitive or more susceptible when used herein may, vice versa, be seen as “less tolerant” or “less resistant”. Similarly, “less tolerant” or “less resistant” may, vice versa, be seen as “more sensitive” or “more susceptible”.

It is generally preferred that the B. vulgaris plants of the present invention and parts thereof are agronomically exploitable. “Agronomically exploitable” means that the B. vulgaris plants and parts thereof are useful for agronomical purposes. For example, the B. vulgaris plants should serve for the purpose of being useful for sugar production, bio fuel production (such as biogas, biobutanol), ethanol production, betaine and/or uridine production. The term “agronomically exploitable” when used herein also includes that the B. vulgaris plants of the present invention are preferably less sensitive against an ALS-inhibitor herbicide, more preferably it is at least 100 times less sensitive, more preferably, 500 times, even more preferably 1000 times and most preferably less than 2000 times. The ALS inhibitor herbicide is one or more described herein, preferably it is foramsulfuron either alone or in combination with one or more further ALS-inhibitor herbicide(s) either from the sub-class of the sulfonylurea herbicides or any other sub-class of the ALS-inhibitor herbicides, most preferably it is foramsulfuron in combination with a further sulfonylurea herbicide and/or an ALS-inhibitor of the sulfonylaminocarbonyltriazolinone herbicide sub-class.

Preferably, agronomically exploitable B. vulgaris plants, most preferably sugar beet plants, of the present invention are fully fertile, more preferably have wild-type fertility. Fertility is of utmost importance for a B. vulgaris plant of the present invention in order to be agronomically exploitable.

An example for an agronomically exploitable B. vulgaris plant is sugar beet. A sugar beet plant of the present invention when cultivated in an area of one hectare yields (about 80,000 to 90,000 sugar beets) should preferably serve for the production of at least 4 tons of sugar. Alternatively, a sugar beet plant of the present invention should preferably contain a sugar content between 15-20%, preferably at least 17% so as to be agronomically exploitable. Thus, sugar beet plants that contain a sugar content between 15-20%, preferably at least 17% are a preferred embodiment of the present invention.

Herbicidal compounds belonging to the class of ALS inhibitors, which can be used in certain embodiments of the invention include (a) sulfonylurea herbicides (Beyer E. M et al. (1988), Sulfonylureas in Herbicides: Chemistry, Degradation, and Mode of Action; Marcel Dekker, New York, 1988, 117-189), (b) sulfonylaminocarbonyltriazolinone herbicides (Pontzen, R., Pflanz.-Nachrichten Bayer, 2002, 55, 37-52), (c) imidazolinone herbicides (Shaner, D. L., et al., Plant Physiol., 1984, 76, 545-546; Shaner, D. L., and O'Connor, S. L. (Eds.) The Imidazolinone Herbicides, CRC Press, Boca Rato, FL, 1991), (d) triazolopyrimidine herbicides (Kleschick, W. A. et al., Agric. FoodChem, 1992, 40, 1083-1085), and (e) pyrimidinyl(oxy/thio)benzoate herbicides (Shimizu, T. J., Pestic. Sci., 1997, 22, 245-256; Shimizu, T. et al., Acetolactate Synthase Inhibitors in Herbicide Classes in Development, Boger, P., Wakabayashi. K., Hirai, K., (Eds.), Springer Verlag, Berlin, 2002, 1-41).

In certain embodiments, the ALS inhibitor is selected from sulfonylurea, sulfonylaminocarbonyltriazolinone, triazolopyrimidine, sulfonanilide, imidazolinone, pyrimidinyloxybenzoeacid, pyrimidinylthiobenzoeacid. Further ALS inhibitors which may be used in certain aspects of the invention are described for instance in WO 2014/090760, WO 2012/049268, WO 2012/049266, EP 2 627 183, and WO 2014/091021, each of which incorporated herein by reference in their entirety.

In certain embodiments, the ALS inhibitor is selected from the ALS inhibitors listed in claims 2-4 of WO2012/049266, all of which are explicitly incorporated herein by reference.

Compounds from the group of the (sulfon)amides are already known as herbicidally active compounds for controlling unwanted vegetation; see, for example, EP 239414, U.S. Pat. No. 4,288,244, DE 3303388, U.S. Pat. Nos. 5,457,085, 3,120,434, 3,480,671, EP 206251, EP 205271, U.S. Pat. Nos. 2,556,664, 3,534,098, EP 53011, U.S. Ser. No. 04/385,927, EP 348737, DE 2822155, U.S. Pat. No. 3,894,078, GB 869169, EP 447004, DE 1039779, HU 176582, U.S. Pat. No. 3,442,945, DE 2305495, DE 2648008, DE 2328340, DE 1014380, HU 53483, U.S. Pat. No. 4,802,907, GB 1040541, U.S. Pat. Nos. 2,903,478, 3,177,061, 2,695,225, DE 1567151, GB 574995, DE 1031571, U.S. Pat. No. 3,175,897, JP 1098331, U.S. Pat. No. 2,913,327, WO 8300329, JP 80127302, DE 1300947, DE 2135768, U.S. Pat. Nos. 3,175,887, 3,836,524, JP 85067463, U.S. Pat. Nos. 3,582,314, 53,330,821, EP 131258, U.S. Pat. Nos. 4,746,353, 4,420,325, 4,394,506, 4,127,405, 4,479,821, 5,009,699, EP 136061, EP 324569, EP 184385, WO 2002030921, WO 09215576, WO 09529899, U.S. Pat. No. 4,668,277, EP 305939, WO 09641537, WO 09510507, EP 7677, CN 01080116, U.S. Pat. No. 4,789,393, EP 971902, U.S. Pat. No. 5,209,771, EP 84020, EP 120814, EP 87780, WO 08804297, EP 5828924, WO 2002036595, U.S. Pat. No. 5,476,936, WO 2009/053058 and the literature cited in the publications mentioned above.

Compounds from the group of the imidazolinones are already known as herbicidally active compounds for controlling unwanted vegetation; see, for example Proc. South. Weed Sci. Soc. 1992. 45, 341, Proc. South. Weed Sci. Soc. Annu. Mtg. 36th, 1983, 29, Weed Sci. Soc. Annu. Mtg. 36th, 1983, 90-91, Weed Sci. Soc. Mtg., 1984, 18, Modern Agrochemicals, 2004, 14-15.

Compounds from the group of the pyrimidinyl(thio)benzoates are already known as herbicidally active compounds for controlling unwanted vegetation; see, for example U.S. Pat. No. 4,906,285, EP 658549, U.S. Pat. No. 5,118,339, WO 91/05781, U.S. Pat. No. 4,932,999, and EP 315889.

Compounds from the group of the sulfonamides are already known as herbicidally active compounds for controlling unwanted vegetation; see, for example WO 93/09099, WO 2006/008159, and WO 2005/096818.

All publications and patents cited in this disclosure are incorporated by reference in their entirety.

In certain embodiments, suitable mutated ALS conferring resistance to ALS inhibitors are as described in EP 2 931 902 and WO 2012/049268, which are incorporated herein in their entirety by reference.

In certain embodiments, the ALS inhibitor herbicide as used herein selected from (sulfon)amides such as sulfonylureas, sulfonylaminocarbonyltriazolinones, sulfonanilides, or triazolopyrimidines; imidazolinones; and pyrimidinyl(thio/oxy)benzoates, preferably selected from sulfonylureas, sulfonylaminocarbonyltriazolinones, imidazolinones, and pyrimidinyl(thio/oxy)benzoates. These classes of ALS inhibitor herbicides can be classified in groups A (with subgroups A1, A2, A3, and A4) B (B1), and C (with subgroups C1 and C2).

In certain embodiments, the ALS inhibitor herbicide as used herein is a sulfonylurea selected from one or more of
amidosulfuron [CAS RN 120923-37-7] (=A1-1);
azimsulfuron [CAS RN 120162-55-2] (=A1-2);
bensulfuron-methyl [CAS RN 83055-99-6] (=A1-3);
chlorimuron-ethyl [CAS RN 90982-32-4] (=A1-4);
chlorsulfuron [CAS RN 64902-72-3] (=A1-5);
cinosulfuron [CAS RN 94593-91-6] (=A1-6);
cyclosulfamuron [CAS RN 136849-15-5] (=A1-7);
ethametsulfuron-methyl [CAS RN 97780-06-8] (=A1-8);
ethoxysulfuron [CAS RN 126801-58-9] (=A1-9);
flazasulfuron [CAS RN 104040-78-0] (=A1-10);
flucetosulfuron [CAS RN 412928-75-7] (=A1-11);
flupyrsulfuron-methyl-sodium [CAS RN 144740-54-5] (=A1-12);
foramsulfuron [CAS RN 173159-57-4] (=A1-13);
halosulfuron-methyl [CAS RN 100784-20-1] (=A1-14);
imazosulfuron [CAS RN 122548-33-8] (=A1-15);
iodosulfuron-methyl-sodium [CAS RN 144550-36-7] (=A1-16);
mesosulfuron-methyl [CAS RN 208465-21-8] (=A1-17);
metsulfuron-methyl [CAS RN 74223-64-6] (=A1-18);
monosulfuron [CAS RN 155860-63-2] (=A1-19);
nicosulfuron [CAS RN 111991-09-4] (=A1-20);
orthosulfamuron [CAS RN 213464-77-8] (=A1-21);
oxasulfuron [CAS RN 144651-06-9] (=A1-22);
primisulfuron-methyl [CAS RN 86209-51-0] (=A1-23);
prosulfuron [CAS RN 94125-34-5] (=A1-24);
pyrazosulfuron-ethyl [CAS RN 93697-74-6] (=A1-25);
rimsulfuron [CAS RN 122931-48-0] (=A1-26);
sulfometuron-methyl [CAS RN 74222-97-2] (=A1-27);
sulfosulfuron [CAS RN 141776-32-1] (=A1-28);
thifensulfuron-methyl [CAS RN 79277-27-3] (=A1-29);
triasulfuron [CAS RN 82097-50-5] (=A1-30);
tribenuron-methyl [CAS RN 101200-48-0] (=A1-31);
trifloxysulfuron [CAS RN 145099-21-4] (sodium) (=A1-32);
triflusulfuron-methyl [CAS RN 126535-15-7] (=A1-33);
tritosulfuron [CAS RN 142469-14-5] (=A1-34);
NC-330 [CAS RN 104770-29-8] (=A1-35);
NC-620 [CAS RN 868680-84-6] (=A1-36);
TH-547 [CAS RN 570415-88-2] (=A1-37);
monosulfuron-methyl [CAS RN 175076-90-1] (=A1-38);
2-iodo-N-[(4-methoxy-6-methyl-1,3,5-triazinyl)carbamoyl]benzene-sulfonamide (=A1-39);
a compound of the general formula (I)

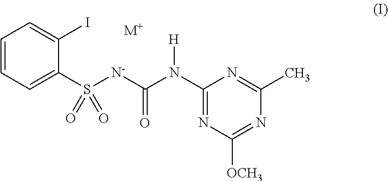

where M⁺ denotes the respective salt of the compound (1), i.e.
its lithium salt (=A1-40); its sodium salt (=A1-41); its potassium salt (=A1-42); its magnesium salt (=A1-43); its calcium salt (=A1-44); its ammonium salt (=A1-45); its methylammonium salt (=A1-46); its dimethylammonium salt (=A1-47); its tetramethylammonium salt (=A1-48); its ethylammonium salt (=A1-49); its diethylammonium salt (=A1-50); its tetraethylammonium salt (=A1-51); its propylammonium salt (=A1-52); its tetrapropylammonium salt (=A1-53); its isopropylammonium salt (=A1-54); its diisopropylammonium salt (=A1-55); its butylammonium salt (=A1-56); its tetrabutylammonium salt (=A1-57); its (2-hydroxyeth-1-yl)ammonium salt (=A1-58); its bis-N,N-(2-hydroxyeth-1-yl)ammonium salt (=A1-59); its tris-N,N,N-(2-hydroxyeth-1-yl)ammonium salt (=A1-60); its 1-phenylethylammonium salt (=A1-61); its 2-phenylethylammonium salt (=A1-62); its trimethylsulfonium salt (=A1-63); its trimethyloxonium salt (=A1-64); its pyridinium salt (=A1-65); its 2-methylpyridinium salt (=A1-66); its 4-methylpyridinium salt (=A1-67); its 2,4-dimethylpyridinium salt (=A1-68); its 2,6-dimethylpyridinium salt (=A1-69); its piperidinium salt (=A1-70); its imidazolium salt (=A1-71); its morpholinium salt (=A1-72); its 1,5-diazabicyclo[4.3.0]non-7-enium salt (=A1-73); its 1,8-diazabicyclo[5.4.0]undec-7-enium salt (=A1-74);
a compound of the formula (II) or salts thereof

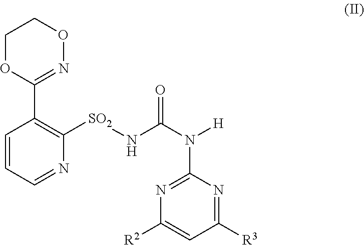

with R², and R³ having the meaning as defined in the below table

| Compound | R² | R³ |
|---|---|---|
| A1-75 | OCH₃ | OC₂H₅ |
| A1-76 | OCH₃ | CH₃ |
| A1-77 | OCH₃ | C₂H₅ |
| A1-78 | OCH₃ | CF₃ |
| A1-79 | OCH₃ | OCF₂H |
| A1-80 | OCH₃ | NHCH₃ |
| A1-81 | OCH₃ | N(CH₃)₂ |
| A1-82 | OCH₃ | Cl |
| A1-83 | OCH₃ | OCH₃ |
| A1-84 | OC₂H₅ | OC₂H₅ |
| A1-85 | OC₂H₅ | CH₃ |
| A1-86 | OC₂H₅ | C₂H₅ |

a compound of formula (III) (=A1-87), i.e. the sodium salt of compound (A1-83)

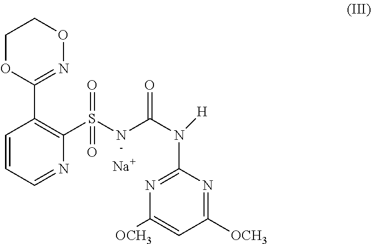 [SMILES: COc1cc(OC)nc(NC(=O)NS(=O)(=O)c2ncccc2C2=NOCCO2)n1 ; [Na+]]

and the compound of formula (IV) (=A1-88), i.e. the sodium salt of compound (A1-82)

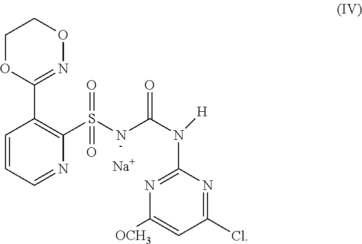 [SMILES: COc1cc(Cl)nc(NC(=O)NS(=O)(=O)c2ncccc2C2=NOCCO2)n1 ; [Na+]]

In certain embodiments, the ALS inhibitor herbicide as used herein is a sulfonylaminocarbonyltriazolinone selected from one or more of
flucarbazone-sodium [CAS RN 181274-17-9] (=A2-1);
propoxycarbazone-sodium [CAS RN 181274-15-7] (=A2-2); and
thiencarbazone-methyl [CAS RN 317815-83-1] (=A2-3).

In certain embodiments, the ALS inhibitor herbicide as used herein is a triazolopyrimidine selected from one or more of
cloransulam-methyl [147150-35-4] (=A3-1);
diclosulam [CAS RN 145701-21-9] (=A3-2);
florasulam [CAS RN 145701-23-1] (=A3-3);
flumetsulam [CAS RN 98967-40-9] (=A3-4);
metosulam [CAS RN 139528-85-1] (=A3-5);
penoxsulam [CAS RN 219714-96-2] (=A3-6);
pyroxsulam [CAS RN 422556-08-9] (=A3-7).

In certain embodiments, the ALS inhibitor herbicide as used herein is a sulfonanilide selected from one or more of
compounds or salts thereof from the group described by the general formula (V):

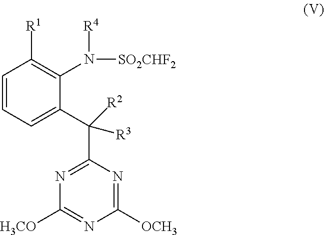

in which
R¹ is halogen, preferably fluorine or chlorine,
R¹ is hydrogen and R³ is hydroxyl or
R² and R³ together with the carbon atom to which they are attached are a carbonyl group
C═O and
R⁴ is hydrogen or methyl;
and more especially compounds of the below given chemical structure (A4-1) to (A4-8)

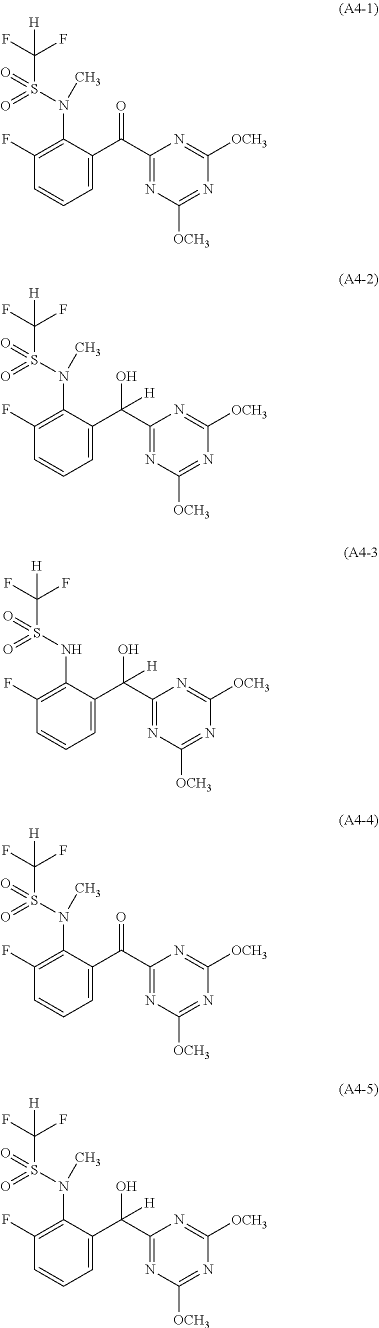

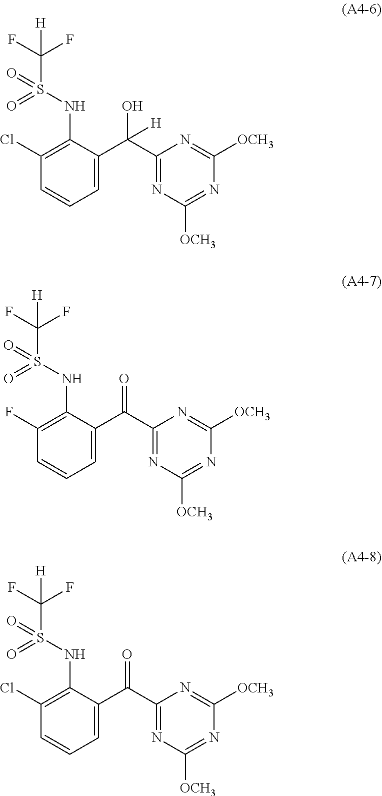 [SMILES: COc1nc(OC)nc(C(O)c2cccc(Cl)c2NS(=O)(=O)C(F)F)n1 ; COc1nc(OC)nc(C(=O)c2cccc(F)c2NS(=O)(=O)C(F)F)n1 ; COc1nc(OC)nc(C(=O)c2cccc(Cl)c2NS(=O)(=O)C(F)F)n1]

In certain embodiments, the ALS inhibitor herbicide as used herein is an imidazolinone selected from one or more of
imazamethabenzmethyl [CAS RN 81405-85-8] (=E1-1);
imazamox [CAS RN 114311-32-9] (=B1-2);
imazapic [CAS RN 104098-48-8] (=B1-3);
imazapyr [CAS RN 81334-34-1] (=B1-4);
imazaquin [CAS RN 81335-37-7] (=B1-5);
imazethapyr [CAS RN 81335-77-5] (=B1-6);
SYP-298 [CAS RN 557064-77-4] (=B1-7);
SYP-300 [CAS RN 374718-10-2] (=B1-8).

In certain embodiments, the ALS inhibitor herbicide as used herein is a pyrimidinyloxybenzoate selected from one or more of
bispyribac-sodium [CAS RN 125401-92-5] (=C1-1);
pyribenzoxim [CAS RN 168088-61-7] (=C1-2);
pyriminobac-methyl [CAS RN 136191-64-5] (=C1-3);
pyribambenz-isopropyl [CAS RN 420138-41-6] (=C1-4);
pyribambenz-propyl [CAS RN 420138-40-5] (=C1-5).

In certain embodiments, the ALS inhibitor herbicide as used herein is a pyrimidinylthiobenzoate selected from one or more of
pyriftalid [CAS RN 135186-78-6] (=C2-1);
pyrithiobac-sodium [CAS RN 123343-16-8] (=C2-2).

The “CAS RN” stated in square brackets behind the names (common names) mentioned under groups A to C corresponds to the “chemical abstract service registry number”, a customary reference number which allows the substances named to be classified unambiguously, since the “CAS RN” distinguishes, inter alia, between isomers including stereoisomers.

The terms “ALS-inhibitor herbicide(s)” or simply “ALS-inhibitor(s)” are used interchangeably. As used herein, an “ALS-inhibitor herbicide” or an “ALS inhibitor” is not meant to be limited to single herbicide that interferes with the activity of the ALS enzyme. Thus, unless otherwise stated or evident from the context, an “ALS-inhibitor herbicide” or an “ALS inhibitor” can be a one herbicide or a mixture of two, three, four, or more herbicides known in the art, preferably as specified herein, each of which interferes with the activity of the ALS enzyme.

ALS inhibitor herbicides which are preferably according to this invention belonging to group (A) are:
amidosulfuron [CAS RN 120923-37-7] (=A1-1);
chlorimuron-ethyl [CAS RN 90982-32-4] (=A1-4);
ethametsulfuron-methyl [CAS RN 97780-06-8] (=A1-8);
ethoxysulfuron [CAS RN 126801-58-9] (=A1-9);
flupyrsulfuron-methyl-sodium [CAS RN 144740-54-5] (=A1-12);
foramsulfuron [CAS RN 173159-57-4] (=A1-13);
iodosulfuron-methyl-sodium [CAS RN 144550-36-7] (=A1-16);
mesosulfuron-methyl [CAS RN 208465-21-8] (=A1-17);
metsulfuron-methyl [CAS RN 74223-64-6] (=A1-18);
monosulfuron [CAS RN 155860-63-2] (=A1-19);
nicosulfuron [CAS RN 111991-09-4] (=A1-20);
sulfosulfuron [CAS RN 141776-32-1] (=A1-28);
thifensulfuron-methyl [CAS RN 79277-27-3] (=A1-29);
tribenuron-methyl [CAS RN 101200-48-0] (=A1-31);
2-iodo-N-[(4-methoxy-6-methyl-1,3,5-triazinyl)carbamoyl]benzene-sulfonamide (=A1-39);
2-iodo-N-[(4-methoxy-6-methyl-1,3,5-triazinyl)carbamoyl]benzene-sulfonamide sodium salt (=A1-41);
(A1-83) or its sodium salt (=A1-87);
propoxycarbazone-sodium [CAS RN 181274-15-7] (=A2-2);
thiencarbazone-methyl [CAS RN 317815-83-1] (=A2-3);
florasulam [CAS RN 145701-23-1] (=A3-3);
metosulam [CAS RN 139528-85-1] (=A3-5);
pyroxsulam [CAS RN 422556-08-9] (=A3-7)
(A4-1);
(A4-2); and
(A4-3).

ALS inhibitor herbicides which are especially preferably used according to this invention belonging to group (A) are:
amidosulfuron [CAS RN 120923-37-7] (=A1-1);
foramsulfuron [CAS RN 173159-57-4] (=A1-13);
iodosulfuron-methyl-sodium [CAS RN 144550-36-7] (=A1-16);
2-iodo-N-[(4-methoxy-6-methyl-1,3,5-triazinyl)carbamoyl]benzene-sulfonamide (=A1-39);
2-iodo-N-[(4-methoxy-6-methyl-1,3,5-triazinyl)carbamoyl]benzene-sulfonamide sodium salt (A1-41);
A1-83 or its sodium salt (=A1-87);
thiencarbazone-methyl [CAS RN 317815-83-1] (=A2-3).

Another ALS inhibitor herbicide which is preferably used according to this invention belonging to group (B) is imazamox [CAS RN 114311-32-9] (=B1-2).

Another ALS inhibitor herbicide which is preferably used according to this invention belonging to group (C) is bispyribac-sodium [CAS RN 125401-92-5] (=C1-1).

It is to be further understood that concerning all above defined ALS inhibitor herbicides and where not already specified by the respective CAS RN, all use forms, such as acids, and salts can be applied according to the invention.

Additionally, the ALS inhibitor herbicide(s) to be used according to the invention may comprise further components, for example agrochemically active compounds of a different type of mode of action and/or the formulation auxiliaries and/or additives customary in crop protection, such as agronomically acceptable carriers, or may be used together with these.

In a preferred embodiment, the herbicide combinations to be used according to the invention comprise effective amounts of the ALS inhibitor herbicide(s) belonging to groups (A), (B) and/or (C) and/or have synergistic actions. The synergistic actions can be observed, for example, when applying one or more ALS inhibitor herbicide(s) belonging to groups (A), (B), and/or (C) together, for example as a co-formulation or as a tank mix; however, they can also be observed when the active compounds are applied at different times (splitting). It is also possible to apply the herbicides or the herbicide combinations in a plurality of portions (sequential application), for example pre-emergence applications followed by post-emergence applications or early post-emergence applications followed by medium or late post-emergence applications. Preference is given here to the joint or almost simultaneous application of the ALS-inhibitor herbicides belonging to groups (A), (B) and/or (C) of the combination in question. The synergistic effects permit a reduction of the application rates of the individual ALS inhibitor herbicides, a higher efficacy at the same application rate, the control of species which were as yet uncontrolled (gaps), control of species which are tolerant or resistant to individual ALS inhibitor herbicides or to a number of ALS inhibitor herbicides, an extension of the period of application and/or a reduction in the number of individual applications required and—as a result for the user—weed control systems which are more advantageous economically and ecologically.

The herbicides to be used according to this invention are all acetolactate synthase (ALS) inhibitor herbicides (which might alternatively and interchangeably also be named as “ALS inhibiting herbicides”) and thus inhibit protein biosynthesis in plants. The application rate of the ALS inhibitor herbicides belonging to groups (A), (B) or (C) (as defined above) can vary within a wide range, for example between 0.001 g and 1500 g of ai/ha (ai/ha means here and below “active substance per hectare”=based on 100% pure active compound). Applied at application rates of from 0.001 g to 1500 g of ai/ha, the herbicides belonging to classes A, B and C according to this invention, preferably the compounds A1-1; A1-4; A1-8; A1-9; A1-12; A1-13; A1-16; A1-17; A1-18; A1-19; A1-20; A1-28; A1-29; A1-31; A1-39; A1-41; A1-83; A1-87; A2-2; A2-3; A3-3; A3-5; A3-7, A4-3, control, when used by the pre- and post-emergence method, a relatively wide spectrum of harmful plants, for example of annual and perennial mono- or dicotyledonous weeds, and also of unwanted crop plants (together also defined as “unwanted vegetation”), including for instance also weed beets, or annual beets, or bolters.

In many applications according to the invention, the application rates are generally lower, for example in the range of from 0.001 g to 1000 g of ai/ha, preferably from 0.1 g to 500 g of ai/ha, particularly preferably from 0.5 g to 250 g of ai/ha, and even more preferably 1.0 g to 200 g of ai/ha. In cases where the application of several ALS inhibitor herbicides is conducted, the quantity represents the total quantity of all of the applied ALS inhibitor herbicides.

For example, the combinations according to the invention of ALS inhibitor herbicides (belonging to groups (A), (B) and/or (C)) allow the activity to be enhanced synergistically in a manner which, by far and in an unexpected manner, exceeds the activities which can be achieved using the individual ALS inhibitor herbicides (belonging to groups (A), (B) and/or (C)).

For combinations of ALS inhibitor herbicides, the preferred conditions are illustrated below.

Of particular interest according to present invention is the use of herbicidal compositions having a content of the following ALS inhibitor herbicides:
(A1-1)+(A1-4); (A1-1)+(A1-8); (A1-1)+(A1-9); (A1-1)+(A1-12); (A1-1)+(A1-13); (A1-1)+(A1-16); (A1-1)+(A1-17); (A1-1)+(A1-18); (A1-1)+(A1-19); (A1-1)+(A1-20); (A1-1)+(A1-28); (A1-1)+(A1-29); (A1-1)+(A1-31); (A1-1)+(A1-39); (A1-1)+(A1-41); (A1-1)+(A1-83); (A1-1)+(A1-87); (A1-1)+(A2-2); (A1-1)+(A2-3); (A1-1)+(A3-3); (A1-1)+(A3-5); (A1-1)+(A3-7); (A1-1)+(A4-1); (A1-1)+(A4-2); (A1-1)+(A4-3);
(A1-4)+(A1-8); (A1-4)+(A1-9); (A1-4)+(A1-12); (A1-4)+(A1-13); (A1-4)+(A1-16); (A1-4)+(A1-17); (A1-4)+(A1-18); (A1-4)+(A1-19); (A1-4)+(A1-20); (A1-4)+(A1-28); (A1-4)+(A1-29); (A1-4)+(A1-31); (A1-4)+(A1-39); (A1-4)+(A1-41); (A1-4)+(A1-83); (A1-4)+(A1-87); (A1-4)+(A2-2); (A1-4)+(A2-3); (A1-4)+(A3-3); (A1-4)+(A3-5); (A1-4)+(A3-7); (A1- 4)+(A4-1); (A1-4)+(A4-2); (A1-4)+(A4-3);
(A1-8)+(A1-9); (A1-8)+(A1-12); (A1-8)+(A1-13); (A1-8)+(A1-16); (A1-8)+(A1-17); (A1- 8)+(A1-18); (A1-8)+(A1-19); (A1-8)+(A1-20); (A1-8)+(A1-28); (A1-8)+(A1-29); (A1-8)+(A1-31); (A1-8)+(A1-39); (A1-8)+(A1-41); (A1-8)+(A1-83); (A1-8)+(A1-87); (A1-8)+(A2-2); (A1-8)+(A2-3); (A1-8)+(A3-3); (A1-8)+(A3-5); (A1-8)+(A3-7); (A1-8)+(A4-1); (A1-8)+(A4-2); (A1-8)+(A4-3);
(A1-9)+(A1-12); (A1-9)+(A1-13); (A1-9)+(A1-16); (A1-9)+(A1-17); (A1-9)+(A1-18); (A1- 9)+(A1-19); (A1-9)+(A1-20); (A1-9)+(A1-28); (A1-9)+(A1-29); (A1-9)+(A1-31); (A1-9)+(A1-39); (A1-9)+(A1-41); (A1-9)+(A1-83); (A1-9)+(A1-87); (A1-9)+(A2-2); (A1-9)+(A2-3); (A1-9)+(A3-3); (A1-9)+(A3-5); (A1-9)+(A3-7); (A1-9)+(A4-1); (A1-9)+(A4-2); (A1-9)+(A4-3);
(A1-12)+(A1-13); (A1-12)+(A1-16); (A1-12)+(A1-17); (A1-12)+(A1-18); (A1-12)+(A1-19); (A1-12)+(A1-20); (A1-12)+(A1-28); (A1-12)+(A1-29); (A1-12)+(A1-31); (A1-12)+(A1-39); (A1-12)+(A1-41); (A1-12)+(A1-83); (A1-12)+(A1-87); (A1-12)+(A2-2); (A1-12)+(A2-3); (A1-12)+(A3-3); (A1-12)+(A3-5); (A1-12)+(A3-7); (A1-12)+(A4-1); (A1-12)+(A4-2); (A1-12)+(A4-3);
(A1-13)+(A1-16); (A1-13)+(A1-17); (A1-13)+(A1-18); (A1-13)+(A1-19); (A1-13)+(A1-20); (A1-13)+(A1-28); (A1-13)+(A1-29); (A1-13)+(A1-31); (A1-13)+(A1-39); (A1-13)+(A1-41);
(A1-13)+(A1-83); (A1-13)+(A1-87); (A1-13)+(A2-2); (A1-13)+(A2-3); (A1-13)+(A3-3); (A1-13)+(A3-5); (A1-13)+(A3-7); (A1-13)+(A4-1); (A1-13)+(A4-2); (A1-13)+(A4-3);
(A1-16)+(A1-17); (A1-16)+(A1-18); (A1-16)+(A1-19); (A1-16)+(A1-20); (A1-16)+(A1-28); (A1-16)+(A1-29); (A1-16)+(A1-31); (A1-16)+(A1-39); (A1-16)+(A1-41); (A1-16)+(A1-83); (A1-16)+(A1-87); (A1-16)+(A2-2); (A1-16)+(A2-3); (A1-16)+(A3-3); (A1-16)+(A3-5); (A1-16)+(A3-7); (A1-16)+(A4-1); (A1-16)+(A4-2); (A1-16)+(A4-3);
(A1-17)+(A1-18); (A1-17)+(A1-19); (A1-17)+(A1-20); (A1-17)+(A1-28); (A1-17)+(A1-29); (A1-17)+(A1-31); (A1-17)+(A1-39); (A1-17)+(A1-41); (A1-17)+(A1-83); (A1-17)+(A1-87); (A1-17)+(A2-2); (A1-17)+(A2-3); (A1-17)+(A3-3); (A1-17)+(A3-5); (A1-17)+(A3-7); (A1-17)+(A4-1); (A1-17)+(A4-2); (A1-17)+(A4-3);
(A1-18)+(A1-19); (A1-18)+(A1-20); (A1-18)+(A1-28); (A1-18)+(A1-29); (A1-18)+(A1-31); (A1-18)+(A1-39); (A1-18)+(A1-41); (A1-18)+(A1-83); (A1-18)+(A1-87); (A1-18)+(A2-2); (A1-18)-i-(A2-3); (A1-18)+(A3-3); (A1-18)+(A3-5); (A1-18)+(A3-7); (A1-18)+(A4-1); (A1-18)+(A4-2); (A1-18)+(A4-3);
(A1-19)+(A1-20); (A1-19)-i-(A1-28); (A1-19)+(A1-29); (A1-19)+(A1-31); (A1-19)+(A1-39); (A1-19)+(A1-41); (A1-19)+(A1-83); (A1-19)+(A1-87); (A1-19)+(A2-2); (A1-19)+(A2-3); (A1-19)+(A3-3); (A1-19)+(A3-5); (A1-19)+(A3-7); (A1-19)+(A4-1); (A1-19)+(A4-2); (A1-19)+(A4-3);
(A1-20)+(A1-28); (A1-20)+(A1-29); (A1-20)+(A1-31); (A1-20)+(A1-39); (A1-20)+(A1-41); (A1-20)-i-(A1-83); (A1-20)+(A1-87); (A1-20)+(A2-2); (A1-20)+(A2-3); (A1-20)+(A3-3); (A1-20)+(A3-5); (A1-20)+(A3-7); (A1-20)+(A4-1); (A1-20)+(A4-2); (A1-20)+(A4-3);
(A1-28)+(A1-29); (A1-28)+(A1-31); (A1-28)+(A1-39); (A1-28)+(A1-41); (A1-28)+(A1-83); (A1-28)+(A1-87); (A1-28)+(A2-2); (A1-28)+(A2-3); (A1-28)+(A3-3); (A1-28)+(A3-5); (A1-28)+(A3-7); (A1-28)+(A4-1); (A1-28)+(A4-2); (A1-28)+(A4-3);
(A1-29)+(A1-31); (A1-29)+(A1-39); (A1-29)+(A1-41); (A1-29)+(A1-83); (A1-29)+(A1-87); (A1-29)+(A2-2); (A1-29)-i-(A2-3); (A1-29)+(A3-3); (A1-29)+(A3-5); (A1-29)+(A3-7); (A1-29)+(A4-1); (A1-29)+(A4-2); (A1-29)+(A4-3);
(A1-31)+(A1-39); (A1-31)+(A1-41); (A1-31)+(A1-83); (A1-31)+(A1-87); (A1-31)+(A2-2); (A1-31)+(A2-3); (A1-31)+(A3-3); (A1-31)+(A3-5); (A1-31)+(A3-7); (A1-31)+(A4-1); (A1-31)+(A4-2); (A1-31)+(A4-3);
(A1-39)+(A1-41); (A1-39)+(A1-83); (A1-39)+(A1-87); (A1-39)+(A2-2); (A1-39)+(A2-3); (A1-39)+(A3-3); (A1-39)+(A3-5); (A1-39)+(A3-7); (A1-39)+(A4-1); (A1-39)+(A4-2); (A1-39)+(A4-3);
(A1-41)+(A1-83); (A1-41)+(A1-87); (A1-41)+(A2-2); (A1-41)+(A2-3); (A1-41)+(A3-3); (A1-41)+(A3-5); (A1-41)+(A3-7); (A1-41)+(A4-1); (A1-41)+(A4-2); (A1-41)+(A4-3);
(A1-83)+(A2-2); (A1-83)+(A2-3); (A1-83)+(A3-3); (A1-83)+(A3-5); (A1-83)+(A3-7); (A1- 83)+(A4-1); (A1-83)+(A4-2); (A1-83)+(A4-3);
(A1-87)+(A2-2); (A1-87)+(A2-3); (A1-87)+(A3-3); (A1-87)+(A3-5); (A1-87)+(A3-7); (A1- 87)+(A4-1); (A1-87)+(A4-2); (A1-87)+(A4-3);
(A2-2)+(A2-3); (A2-2)+(A3-3); (A2-2)+(A3-5); (A2-2)+(A3-7); (A2-2)+(A4-1); (A2-2)+(A4-2); (A2-2)+(A4-3);
(A2-3)+(A3-3); (A2-3)+(A3-5); (A2-3)+(A3-7); (A2-3)+(A4-1); (A2-3)+(A4-2); (A2-3)+(A4-3);
(A3-3)+(A3-5); (A3-3)+(A3-7); (A3-3)+(A4-1); (A3-3)+(A4-2); (A3-3)+(A4-3);
(A3-5)+(A3-7); (A3-5)+(A4-1); (A3-5)+(A4-2); (A3-5)+(A4-3);
(A3-7)+(A4-1); (A3-7)+(A4-2); (A3-7)+(A4-3);
(A4-1)+(A4-2); (A4-1)+(A4-3); and
(A4-2)+(A4-3);

Additionally, the ALS inhibitor herbicides to be used according to the invention may comprise further components, for example agrochemically active compounds of a different type of mode of action and/or the formulation auxiliaries and/or additives customary in crop protection, or may be used together with these.

The ALS inhibitor herbicide(s) to be used according to the invention or combinations of various such ALS inhibitor herbicides may furthermore comprise various agrochemically active compounds, for example from the group of the safeners, fungicides, insecticides, or from the group of the formulation auxiliaries and additives customary in crop protection.

In a further embodiment, the invention relates to the use of effective amounts of ALS inhibitor herbicide(s) (i.e. members of the groups (A), (B) and/or (C)) and non-ALS inhibitor herbicides (i.e. herbicides showing a mode of action that is different to the inhibition of the ALS enzyme [acetohydroxyacid synthase; EC 2.2.1.6] (group D herbicides) in order obtain synergistic effect for the control of unwanted vegetation. Such synergistic actions can be observed, for example, when applying one or more ALS inhibitor herbicides (i.e. members of the groups (A), (B), and/or (C)) and one or more non-ALS inhibitor herbicides (group D herbicides) together, for example as a co-formulation or as a tank mix; however, they can also be observed when the active compounds are applied at different times (splitting). It is also possible to apply the ALS inhibitor herbicides and non-ALS inhibitor herbicides in a plurality of portions (sequential application), for example pre-emergence applications followed by post-emergence applications or early post-emergence applications followed by medium or late post-emergence applications. Preference is given here to the joint or almost simultaneous application of the herbicides ((A), (B) and/or (C)) and (D) of the combination in question.

Suitable partner herbicides to be applied together with ALS inhibitor herbicides are, for example, the following herbicides which differ structurally from the herbicides belonging to the groups (A), (B), and (C) as defined above, preferably herbicidally active compounds whose action is based on inhibition of, for example, acetyl coenzyme A carboxylase, PS I, PS II, HPPDO, phytoene desaturase, protoporphyrinogen oxidase, glutamine synthetase, cellulose biosynthesis, 5-enolpyruvylshikimate 3-phosphate synthetase, as described, for example, in Weed Research 26, 441-445 (1986), or “The Pesticide Manual”, 14th edition, The British Crop Protection Council, 2007, or 15th edition 2010, or in the corresponding “e-Pesticide Manual”, Version 5 (2010), in each case published by the British Crop Protection Council, (hereinbelow in short also “PM”), and in the literature cited therein. Lists of common names are also available in “The Compendium of Pesticide Common Names” on the internet. Herbicides known from the literature (in brackets behind the common name hereinafter also classified by the indicators D1 to D426), which can be combined with ALS-inhibitor herbicides of groups (A), (B) and/or (C) and to be used according to present invention are, for example, the active compounds listed below: (note: the herbicides are referred to either by the “common name” in accordance with the International Organization for Standardization (ISO) or by the chemical name, together where appropriate with a customary code number, and in each case include all use forms, such as acids, salts, esters and isomers, such as stereoisomers and optical isomers, in particular the commercial form or the commercial forms, unless the context indicates otherwise. The citation given is of one use form and in some cases of two or more use forms):
acetochlor (=D1), acibenzolar (=D2), acibenzolar-S-methyl (=D3), acifluorfen (=D4), acifluorfen-sodium (=D5), aclonifen (=D6), alachlor (=D7), allidochlor (=D8), alloxydim (=D9), alloxydim-sodium (=D10), ametryn (=D11), amicarbazone (=D12), amidochlor (=D13), aminocyclopyrachlor (=D14), aminopyralid (=D15), amitrole (=D16), ammonium sulfamate (=D17), ancymidol (=D18), anilofos (=D19), asulam (=D20), atrazine (=D21), azafenidin (=D22), aziprotryn (=D23), beflubutamid (=D24), benazolin (=D25), benazolin-ethyl (=D26), bencarbazone (=D27), benfluralin (=D28), benfuresate (=D29), bensulide (=D30), bentazone (=D31), benzfendizone (=D32), benzobicyclon (=D33), benzofenap (=D34), benzofluor (=D35), benzoylprop (=D36), bicyclopyrone (=D37), bifenox (=D38), bilanafos (=D39), bilanafos-sodium (=D40), bromacil (=D41), bromobutide (=D42), bromofenoxim (=D43), bromoxynil (=D44), bromuron (=D45), buminafos (=D46), busoxinone (=D47), butachlor (=D48), butafenacil (=D49), butamifos (=D50), butenachlor (=D51), butralin (=D52), butroxydim (=D53), butylate (=D54), cafenstrole (=D55), carbetamide (=D56), carfentrazone (=D57), carfentrazone-ethyl (=D58), chlomethoxyfen (=D59), chloramben (=D60), chlorazifop (=D61), chlorazifop-butyl (=D62), chlorbromuron (=D63), chlorbufam (=D64), chlorfenac (=D65), chlorfenac-sodium (=D66), chlorfenprop (=D67), chlorflurenol (=D68), chlorflurenol-methyl (=D69), chloridazon (=D70), chlormequat-chloride (=D71), chlornitrofen (=D72), chlorophthalim (=D73), chlorthal-dimethyl (=D74), chlorotoluron (=D75), cinidon (=D76), cinidon-ethyl (=D77), cinmethylin (=D78), clethodim (=D79), clodinafop (=D80), clodinafop-propargyl (=D81), clofencet (=D82), clomazone (=D83), clomeprop (=D84), cloprop (=D85), clopyralid (=D86), cloransulam (=D87), cloransulam-methyl (=D88), cumyluron (=D89), cyanamide (=D90), cyanazine (=D91), cyclanilide (=D92), cycloate (=D93), cycloxydim (=D94), cycluron (=D95), cyhalofop (=D96), cyhalofop-butyl (=D97), cyperquat (=D98), cyprazine (=D99), cyprazole (=D100), 2,4-D (=D101), 2,4-DB (=D102), daimuron/dymron (=D103), dalapon (=D104), daminozide (=D105), dazomet (=D106), n-decanol (=D-107), desmedipham (=D108), desmetryn (=D109), detosyl-pyrazolate (=D110), diallate (=D111), dicamba (=D112), dichlobenil (=D113), dichlorprop (=D114), dichlorprop-P (=D115), diclofop (=D116), diclofop-methyl (=D117), diclofop-P-methyl (=D118), diethatyl (=D119), diethatyl-ethyl (=D120), difenoxuron (=D121), difenzoquat (=D122), diflufenican (=D123), diflufenzopyr (=D124), diflufenzopyr-sodium (=D125), dimefuron (=D126), dikegulac-sodium (=D127), dimefuron (=D128), dimepiperate (=D129), dimethachlor (=D130), dimethametryn (=D131), dimethenamid (=D132), dimethenamid-P (=D133), dimethipin (=D134), dimetrasulfuron (=D135), dinitramine (=D136), dinoseb (=D137), dinoterb (=D138), diphenamid (=D139), dipropetryn (=D140), diquat (=D141), diquat-dibromide (=D142), dithiopyr (=D143), diuron (=D144), DNOC (=D145), eglinazine-ethyl (=D146), endothal (=D147), EPTC (=D148), esprocarb (=D149), ethalfluralin (=D150), ethephon (=D151), ethidimuron (=D152), ethiozin (=D153), ethofumesate (=D154), ethoxyfen (=D155), ethoxyfen-ethyl (=D156), etobenzanid (=D157), F-5331 (=2-Chlor-4-fluor-5-[4-(3-fluorpropyl)-4,5-dihydro-5-oxo-1H-tetrazol-1-yl]-phenyl]-ethansulfonamid) (=D158), F-7967 (=3-[7-Chlor-5-fluor-2-(trifluormethyl)-1H-benzimidazol-4-yl]-1-methyl-6-(trifluormethyl)pynmidin-2,4(1H,3H)-dion) (=D159), fenoprop (=D160), fenoxaprop (=D161), fenoxaprop-P (=D162), fenoxaprop-ethyl (=D163), fenoxaprop-P-ethyl (=D164), fenoxasulfone (=D165), fentrazamide (=D166), fenuron (=D167), flamprop (=D168), flamprop-M-isopropyl (=D169), flam prop-M-methyl (=D170), fluazifop (=D171), fluazifop-P (=D172), fluazifop-butyl (=D173), fluazifop-P-butyl (=D174), fluazolate (=D175), fluchloralin (=D176), flufenacet (thiafluamide) (=D177), flufenpyr (=D178), flufenpyr-ethyl (=D179), flumetralin (=D180), flumiclorac (=D181), flumiclorac-pentyl (=D182), flumioxazin (=D183), flumipropyn (=D184), fluometuron (=D185), fluorodifen (=D186), fluoroglycofen (=D187), fluoroglycofen-ethyl (=D188), flupoxam (=D189), flupropacil (=D190), flupropanate (=D191), flurenol (=D192), flurenol-butyl (=D193), fluridone (=D194), flurochloridone (=D195), fluroxypyr (=D196), fluroxypyr-meptyl (=D197), flurprimidol (=D198), flurtamone (=D199), fluthiacet (=D200), fluthiacet-methyl (=D201), fluthiamide (=D202), fomesafen (=203), forchlorfenuron (=D204), fosamine (=D205), furyloxyfen (=D206), gibberellic acid (=D207), glufosinate (=D208), glufosinate-ammonium (=D209), glufosinate-P (=D210), glufosinate-P-ammonium (=D211), glufosinate-P-sodium (=D212), glyphosate (=D213), glyphosate-isopropylammonium (=D214), H-9201 (=O-(2,4-Dimethyl-6-nitrophenyl)-O-ethyl-isopropylphosphoramidothioat) (=D215), halosafen (=D216), haloxyfop (=D217), haloxyfop-P (=D218), haloxyfop-ethoxyethyl (=D219), haloxyfop-P-ethoxyethyl (=D220), haloxyfop-methyl (=D221), haloxyfop-P-methyl (=D222), hexazinone (=D223), HW-02 (=1-(Dimethoxyphosphoryl)-ethyl(2,4-dichlorphenoxy)acetate) (=D224), inabenfide (=D225), indanofan (=D226), indaziflam (=D227), indol-3-acetic acid (IAA) (=D228), 4-indol-3-ylbutyric acid (IBA) (=D229), ioxynil (=D230), ipfencarbazone (=D231), isocarbamid (=D232), isopropalin (=D233), isoproturon (=D234), isouron (=D235), isoxaben (=D236), isoxachlortole (=D237), isoxaflutole (=D238), isoxapyrifop (=D239), KUH-043 (=3-({[5-(Difluormethyl)-1-methyl-3-(trifluormethyl)-1H-pyrazol-4-yl]methyl}sulfonyl)-5,5-dimethyl-4,5-dihydro-1,2-oxazol) (=D240), karbutilate (=D241), ketospiradox (=D242), lactofen (=D243), lenacil (=D244), linuron (=D245), maleic hydrazide (=D246), MCPA (=D247), MCPB (=D248), MCPB-methyl, -ethyl and -sodium (=D249), mecoprop (=D250), mecoprop-sodium (=D251), mecoprop-butotyl (=D252), mecoprop-P-butotyl (=D253), mecoprop-P-dimethylammonium (=D254), mecoprop-P-2-ethylhexyl (=D255), mecoprop-P-potassium (=D256), mefenacet (=D257), mefluidide (=D258), mepiquat-chloride (=D259), mesotrione (=D260), methabenzthiazuron (=D261), metam (=D262), metamifop (=D263), metamitron (=D264), metazachlor (=D265), metazole (=D266), methiopyrsulfuron (=D267), methiozolin (=D268), methoxyphenone (=D269), methyldymron (=D270), 1-methylcyclopropen (=D271), methylisothiocyanat (=D272), metobenzuron (=D273), metobromuron (=D274), metolachlor (=D275), S-metolachlor (=D-276), metoxuron (=D277), metribuzin (=D278), molinate (=D279), monalide (=D280), monocarbamide (=D281), monocarbamide-dihydrogensulfate (=D282), monolinuron (=D283), monosulfuron-ester (=D284), monuron (=D285), MT-128 (=6-Chlor-N-[(2E)-3-chlorprop-2-en-1-yl]-5-methyl-N-phenylpyridazin-3-amine) (=D286), MT-5950 (=N-[3-Chlor-4-(1-methylethyl)-phenyl]-2-methylpentanamide) (=D287), NGGC-011 (=D288), naproanilide (=D289), napropamide (=D290), naptalam (=D291), NC-310 (=4-(2,4-Dichlorobenzoyl)-1-methyl-5-benzyloxypyrazole) (=D292), neburon (=D293), nipyraclofen (=D294), nitralin (=D295), nitrofen (=D296), nitrophenolat-sodium (isomer mixture) (=D297), nitrofluorfen (=D298), nonanoic acid (=D299), norflurazon (=D300), orbencarb (=D301), oryzalin (=D302), oxadiargyl (=D303), oxadiazon (=D304), oxaziclomefone (=D305), oxyfluorfen (=D306), paclobutrazol (=D307), paraquat (=D308), paraquat-dichloride (=D309), pelargonic acid (nonanoic acid) (=D310), pendimethalin (=D311), pendralin (=D312), pentanochlor (=D313), pentoxazone (=D314), perfluidone (=D315), pethoxamid (=D317), phenisopham (=D318), phenmedipham (=D319), phenmedipham-ethyl (=D320), picloram (=D321), picolinafen (=D322), pinoxaden (=D323), piperophos (=D324), pirifenop (=D325), pirifenop-butyl (=D326), pretilachlor (=D327), probenazole (=D328), profluazol (=D329), procyazine (=D330), prodiamine (=D331), prifluraline (=D332), profoxydim (=D333), prohexadione (=D334), prohexadione-calcium (=D335), prohydrojasmone (=D336), prometon (=D337), prometryn (=D338), propachlor (=D339), propanil (=D340), propaquizafop (=D341), propazine (=D342), propham (=D343), propisochlor (=D344), propyzamide (=D345), prosulfalin (=D346), prosulfocarb (=D347), prynachlor (=D348), pyraclonil (=D349), pyraflufen (=D350), pyraflufen-ethyl (=D351), pyrasulfotole (=D352), pyrazolynate (pyrazolate) (=D353), pyrazoxyfen (=D354), pyribambenz (=D355), pyributicarb (=D356), pyridafol (=D357), pyridate (=D358), pyriminobac (=D359), pyrimisulfan (=D360), pyroxasulfone (=D361), quinclorac (=D362), quinmerac (=D363), quinoclamine (=D364), quizalofop (=D365), quizalofop-ethyl (=D366), quizalofop-P (=D367), quizalofop-P-ethyl (=D368), quizalofop-P-tefuryl (=D369), saflufenacil (=D370), secbumeton (=D371), sethoxydim (=D372), siduron (=D373), simazine (=D374), simetryn (=D375), SN-106279 (=Methyl-(2R)-2-({7-[2-chlor-4-(trifluormethyl)phenoxy]-2-naphthyl}oxy)-propanoate) (=D376), sulcotrione (=D377), sulfallate (CDEC) (=D378), sulfentrazone (=D379), sulfosate (glyphosate-trimesium) (=D380), SYN-523 (=D381), SYP-249 (=1-Ethoxy-3-methyl-1-oxobut-3-en-2-yl-5-[2-chlor-4-(trifluormethyl)phenoxy]-2-nitrobenzoate) (=D382), tebutam (=D383), tebuthiuron (=D384), tecnazene (=D385), tefuryltrione (=D386), tembotrione (=D387), tepraloxydim (=D388), terbacil (=D389), terbucarb (=D390), terbuchlor (=D391), terbumeton (=D392), terbuthylazine (=D393), terbutryn (=D394), thenylchlor (=D395), thiafluamide (=D396), thiazafluron (=D397), thiazopyr (=D398), thidiazimin (=D399), thidiazuron (=D400), thiobencarb (=D401), tiocarbazil (=D402), topramezone (=D403), tralkoxydim (=D404), triallate (=D405), triaziflam (=D406), triazofenamide (=D407), trichloracetic acid (TCA) (=D408), triclopyr (=D409), tridiphane (=D410), trietazine (=D411), trifluralin (=D412), trimeturon (=D413), trinexapac (=D414), trinexapac-ethyl (=D415), tsitodef (=D416), uniconazole (=D417), uniconazole-P (=D418), vernolate (=D419), ZJ-0862 (=3,4-Dichlor-N-{2-[(4,6-dimethoxypyrimidin-2-yl)oxy]benzyl}aniline) (=D420), and the below compounds defined by their chemical structure, respectively:

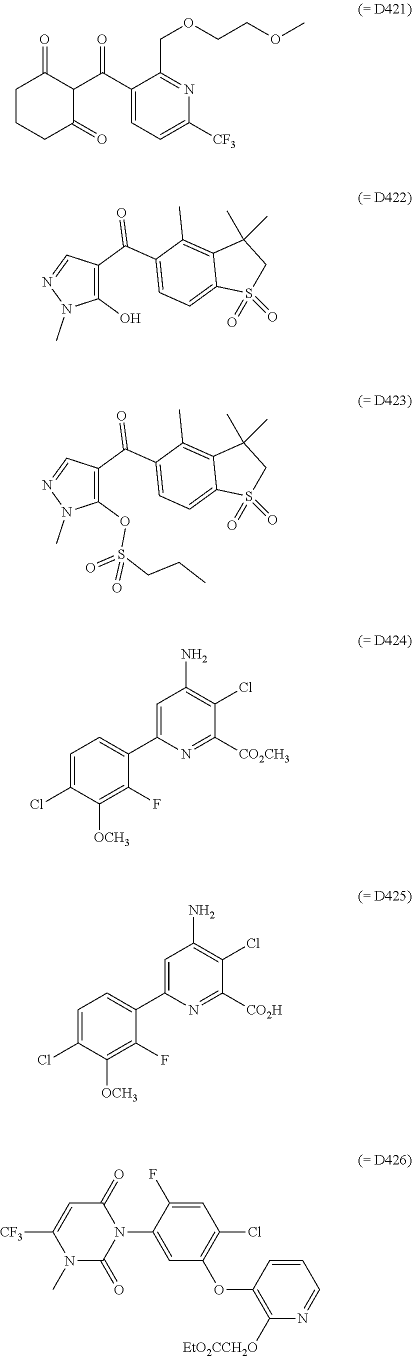

Preferably, further herbicides which differ structurally and via their mode of action from the ALS inhibitor herbicides belonging to the groups (A), (B), and (C) as defined above and to be applied according to the present invention are those belonging to the group of: chloridazon (=D70), clethodim (=D79), clodinafop (=D80), clodinafop-propargyl (=D81), clopyralid (=D86), cycloxydim (=D94), desmedipham (=D108), dimethenamid (=D132), dimethenamid-P (=D133), ethofumesate (=D154), fenoxaprop (=D161), fenoxaprop-P (=D162), fenoxaprop-ethyl (=D163), fenoxaprop-P-ethyl (=D164), fluazifop (=D171), fluazifop-P (=D172), fluazifop-butyl (=D173), fluazifop-P-butyl (=D174), glufosinate (=D208), glufosinate-ammonium (=D209), glufosinate-P (=D210), glufosinate-P-ammonium (=D211), glufosinate-P-sodium (=D212), glyphosate (=D213), glyphosate-isopropylammonium (=D214), haloxyfop (=D217), haloxyfop-P (=D218), haloxyfop-ethoxyethyl (=D219), haloxyfop-P-ethoxyethyl (=D220), haloxyfop-methyl (=D221), haloxyfop-P-methyl (=D222), lenacil (=D244), metamitron (=D264), phenmedipham (=D319), phenmedipham-ethyl (=D320), propaquizafop (=D341), quinmerac (=D363), quizalofop (=D365), quizalofop-ethyl (=D366), quizalofop-P (=D367), quizalofop-P-ethyl (=D368), quizalofop-P-tefuryl (=D369), sethoxydim (=D372).

Even more preferably, further herbicides which differ from the ALS inhibitor herbicides belonging to the groups (A), (B), and (C) as defined above and to be applied according to the invention in connection with ALS inhibitor herbicides belonging to the groups (A), (B), and (C) are those belonging to the group of: desmedipham (=D108), ethofumesate (=D154), glufosinate (=D208), glufosinate-ammonium (=D209), glufosinate-P (=D210), glufosinate-P-ammonium (=D211), glufosinate-P-sodium (=D212), glyphosate (=D213), glyphosate-isopropylammonium (=D214), lenacil (=D244), metamitron (=D264), phenmedipham (=D319), phenmedipham-ethyl (=D320).

Mixtures containing ALS inhibitor herbicides and non-ALS inhibitor herbicides, compositions comprising mixtures of one or more ALS inhibitor herbicide(s) (compounds belonging to one or more of groups (A), (B) and (C)) and non-ALS inhibitor herbicide(s) (group (D) members; as defined above) that are of very particular interest in order to be used according to present invention are:
(A1-1)+(D108); (A1-1)+(D154); (A1-1)+(D208); (A1-1)+(D209); (A1-1)+(D210); (A1-1)+(D212); (A1-1)+(D213); (A1-1)+(D214); (A1-1)+(D244); (A1-1)+(D264); (A1-1)+(D319); (A1-1)+(D320).
(A1-13)+(D108); (A1-13)+(D154); A1-13)+(D208); (A1-13)+(D209); (A1-13)+(D210); (A1-13)+(D212); (A1-13)+(D213); (A1-13)+(D214); (A1-13)+(D244); A1-13)+(D264); A1-13)+(D319); (A1-13)+(D320).
(A1-16)+(D108); A1-16)+(D154); A1-16)+(D208); (A1-16)+(D209); (A1-16)+(D210); (A1-16)+(D212); (A1-16)+(D213); (A1-16)+(D214); (A1-16)+(D244); A1-16)+(D264); A1-16)+(D319); (A1-16)+(D320).
(A1-39)+(D108); A1-39)+(D154); A1-39)+(D208); (A1-39)+(D209); (A1-39)+(D210); (A1-39)+(D212); (A1-39)+(D213); (A1-39)+(D214); (A1-39)+(D244); A1-39)+(D264); A1-39)+(D319); (A1-39)+(D320).
(A1-41)+(D108); A1-41)+(D154); A1-41)+(D208); (A1-41)+(D209); (A1-41)+(D210); (A1-41)+(D212); (A1-41)+(D213); (A1-41)+(D214); (A1-41)+(D244); A1-41)+(D264); A1-41)+(D319); (A1-41)+(D320).
(A1-83)+(D108); A1-83)+(D154); A1-83)+(D208); (A1-83)+(D209); (A1-83)+(D210); (A1-83)+(D212); (A1-83)+(D213); (A1-83)+(D214); (A1-83)+(D244); A1-83)+(D264); A1-83)+(D319); (A1-83)+(D320).
(A1-87)+(D108); A1-87)+(D154); A1-87)+(D208); (A1-87)+(D209); (A1-87)+(D210); (A1-87)+(D212); (A1-87)+(D213); (A1-87)+(D214); (A1-87)+(D244); A1-87)+(D264); A1-87)+(D319); (A1-87)+(D320).
(A2-3)+(D108); (A2-3)+(D154); (A2-3)+(D208); (A2-3)+(D209); (A2-3)+(D210); (A2-3)+(D212); (A2-3)+(D213); (A2-3)+(D214); (A2-3)+(D244); (A2-3)+(D264); (A2-3)+(D319); (A2-3)+(D320).
(B1-2)+(D108); (B1-2)+(D154); (B1-2)+(D208); (B1-2)+(D209); (B1-2)+(D210); (B1-2)+(D212); (B1-2)+(D213); (B1-2)+(D214); (B1-2)+(D244); (B1-2)+(D264); (B1-2)+(D319); (B1-2)+(D320).
(C1-1)+(D108); (C1-1)+(D154); (C1-1)+(D208); (C1-1)+(D209); (C1-1)+(D210); (C1-1)+(D212); (C1-1)+(D213); (C1-1)+(D214); (C1-1)+(D244); (C1-1)+(D264); (C1-1)+(D319); (C1-1)+(D320).

In certain embodiments, non-ALS inhibitor herbicides may be applied in combination with the ALS inhibitor herbicides. In certain embodiments, the application of the respective herbicides (i) takes place jointly or simultaneously, or (ii) takes place at different times and/or in a plurality of portions (sequential application), in pre-emergence applications followed by post-emergence applications or early post-emergence applications followed by medium or late post-emergence applications. In certain embodiments, the herbicides are selected from chloridazon, clethodim, clodinafop, clodinafop-propargyl, clopyralid, cycloxydim, desmedipham, dimethenamid, dimethenamid-P, ethofumesate, fenoxaprop, fenoxaprop-P, fenoxaprop-ethyl, fenoxaprop-P-ethyl, fluazifop, fluazifop-P, fluazifop-butyl, fluazifop-P-butyl, glufosinate, glufosinate-ammonium, glufosinate-P, glufosinate-P-ammonium, glufosinate-P-sodium, haloxyfop, haloxyfop-P, haloxyfop-ethoxyethyl, haloxyfop-P-ethoxyethyl, haloxyfop-methyl, haloxyfop-P-methyl, lenacil, metamitron, phenmedipham, phenmedipham-ethyl, propaquizafop, quinmerac, quizalofop, quizalofop-ethyl, quizalofop-P, quizalofop-P-ethyl, quizalofop-P-tefuryl, sethoxydim.

The application of ALS inhibitor herbicides also act efficiently on perennial weeds which produce shoots from rhizomes, root stocks and other perennial organs and which are difficult to control. Here, the substances can be applied, for example, by the pre-sowing method, the pre-emergence method or the post-emergence method, for example jointly or separately. Preference is given, for example, to application by the post-emergence method, in particular to the emerged harmful plants.

Specific examples may be mentioned of some representatives of the monocotyledonous and dicotyledonous weed flora which can be controlled by the ALS inhibitor herbicides, without the enumeration being restricted to certain species.

Examples of weed species on which the application according to present invention act efficiently are, from amongst the monocotyledonous weed species, Avena spp., Alopecurus spp., Apera spp., Brachiaria spp., Bromus spp., Digitaria spp., Lolium spp., Echinochloa spp., Panicum spp., Phalaris spp., Poa spp., Setaria spp. and also Cyperus species from the annual group, and, among the perennial species, Agropyron, Cynodon, Imperata and Sorghum and also perennial Cyperus species.

In the case of the dicotyledonous weed species, the spectrum of action extends to genera such as, for example, Abutilon spp., Amaranthus spp., Chenopodium spp., Chrysanthemum spp., Galium spp., Ipomoea spp., Kochia spp., Lamium spp., Matricaria spp., Pharbitis spp., Polygonum spp., Sida spp., Sinapis spp., Solanum spp., Stellaria spp., Veronica spp. and Viola spp., Xanthium spp., among the annuals, and Convolvulus, Cirsium, Rumex and Artemisia in the case of the perennial weeds.

The herbicides described herein may also be used to control for instance weed beets (or annual beets). The cultivated Beta vulgaris is a biennial plant which forms a storage root and a leaf rosette in the first year. Shoot elongation (bolting) and flower formation starts after a period of low temperature, whereas many wild beets of the genus B. vulgaris ssp. maritima show an annual growing habit due to the presence of the bolting gene B at the B locus. The BOLTING gene (B gene) is responsible for the determination of the annual habit in sugar beet. Annuality in the Beta species is considered a monogenic and dominant trait. Plants carrying the dominant B allele are able to switch from juvenile to reproductive stages in a vernalization-independent manner, contrary to biennial plants carrying the b allele that obligatory require vernalization for bolting and subsequent flowering to occur. The dominant allele of locus B is abundant in wild beets and causes bolting under long days without the cold requirement usually essential for biennial cultivars carrying the recessive allele. “B gene” as used herein refers to a gene that is responsible for the determination of the annual habit (early bolting) in Beta vulgaris, such as sugar beet. Plants carrying the dominant allele B are able to switch from juvenile to reproductive stages in a vernalization-independent manner, i.e. make shoot elongation followed by flowering without prior exposure to cold temperatures.

In an aspect, the invention relates to a method for controlling unwanted vegetation, such as in Beta vulgaris growing areas, or for maintaining or increasing the yield in Beta vulgaris growing areas, comprising the steps of:
a) planting Beta vulgaris plants or sowing Beta vulgaris seeds according to the invention as described herein elsewhere, in particular comprising an ALS protein having an amino acid at position 371 which is different than aspartic acid,
b) applying one or more ALS inhibitor herbicide to the growing plants, preferably at a dosage sufficient for inhibiting the growth of the unwanted vegetation, more preferably at a dosage sufficient for killing the unwanted vegetation, and
c) optionally, repeating step b) during the growing season.

In a related aspect, the invention relates to the use of one or more ALS inhibitor herbicide, as defined herein elsewhere for controlling unwanted vegetation, such as in Beta vulgaris growing areas, or for maintaining or increasing the yield in Beta vulgaris growing areas, in which the Beta vulgaris plants are according to the invention as described herein elsewhere, in particular comprising an ALS protein having an amino acid at position 371 which is different than aspartic acid.

It is particular preferred that beet root (or root beet) yield is maintained or increased.

In certain embodiments, the methods for controlling unwanted vegetation comprise methods for controlling bolters, weed beets, or annual beets as described herein and may relate to methods for controlling unwanted vegetation, such as bolters, weed beets, or annual beets in Beta vulgaris growing areas, preferably Beta vulgaris subsp. vulgaris growing areas, in particular Beta vulgaris subsp. vulgaris var. altissima growing areas. In certain embodiments, the methods for controlling unwanted vegetation, such as bolters, weed beets, or annual beets as described herein relate to methods for controlling unwanted vegetation, such as bolters, weed beets, or annual beets in biennial Beta vulgaris growing areas, preferably biennial Beta vulgaris subsp. vulgaris growing areas, in particular biennial Beta vulgaris subsp. vulgaris var. altissima growing areas.

In certain embodiments, the uses as described herein relate to uses in Beta vulgaris growing areas, preferably Beta vulgaris subsp. vulgaris growing areas, in particular Beta vulgaris subsp. vulgaris var. altissima growing areas. In certain embodiments, the uses as described herein relate to uses in biennial Beta vulgaris growing areas, preferably biennial Beta vulgaris subsp. vulgaris growing areas, in particular biennial Beta vulgaris subsp. vulgaris var. altissima growing areas.

A “biennial” or “biannual” Beta vulgaris refers to a Beta vulgaris plant that takes two years to complete its biological lifecycle. An “annual” Beta vulgaris refers to a Beta vulgaris plant that germinates, flowers, and dies in one year. An “annual Beta vulgaris” refers to a Beta vulgaris plant containing the dominant allele B at the B locus in a heterozygous or homozygous state.

A “biennial Beta vulgaris” refers to a Beta vulgaris plant containing the recessive allele b at the B locus in a homozygous state “Bolting” refers to the transition from the vegetative rosette stage to the inflorescence or reproductive growth stage, in particular shoot formation. Bolting (stem elongation) is the first step clearly visible in the transition from vegetative to reproductive growth. Bolting can be characterized by an (unwanted) emergence of shoots during the first year of growing, which is disadvantageously in harvesting and processing, but also reduces crop yield. Indeed, bolting and flowering of Beta vulgaris plants is undesirable, since in the case of for instance sugar beets it is not the seeds or fruits, but rather the underground part of the plant, the storage root, that is used, and the energy stored in the root would be consumed during the bolting and flowering of the plant.

As used herein, the term “bolters” refers to Beta vulgaris plants that bolt during the growing season, in particular the same year as the Beta vulgaris plants are planted or sown, preferably before the time the beets are or need to be harvested. In certain embodiments, the bolters are annual Beta vulgaris plants. In certain embodiments, the bolters are weed beets. In certain embodiments, the bolters are sea beets (i.e. Beta vulgaris subsp. maritima). In certain embodiments, the bolters are not Beta vulgaris subsp. vulgaris. In certain embodiments, the bolters are not Beta vulgaris subsp. vulgaris var. altissima. In certain embodiments, the bolters comprise the dominant bolting gene (B gene). As used herein, the term “weed beets” refers to unwanted beet plants, as opposed to the intended cultivated beet plants in the beet growing areas. Weed beets typically are wild beets. Weed beets are preferably annual beets, optionally Beta vulgaris subsp. maritima.

As used herein, “controlling” in the context of controlling unwanted vegetation, such as bolters or unwanted plants or vegetation etc. includes inhibiting or preventing the growth of bolters, weed beets, or annual beets or unwanted plants or inhibiting bolting of weed beets or annual beets, or at least inhibiting seed production of weed beets or annual beets. “Controlling” may also include killing bolters, weed beets or annual beets, or unwanted plants, preferably before bolting occurs, or at least before seed production of the bolters, weed beets, or annual beets. “Controlling” may also include reducing the amount of bolters, weed beets, or annual beets, or unwanted plants in beet growing areas, preferably before bolting occurs, or at least before seed production of the bolters, weed beets, or annual beets. Controlling bolters or unwanted plants etc. in certain embodiments refers to a reduction of at least 50% of the amount of bolters or unwanted plants etc. or a reduction of at least 50% of the biomass of bolters or unwanted plants etc., such as preferably at least 60%, more preferably at least 70%, such as at least 80% or at least 90%.

As used herein, “Unwanted plants” or “unwanted vegetation” are to be understood as meaning all plants which grow in locations where they are unwanted. This can, for example, be harmful plants (for example monocotyledonous or dicotyledonous weeds or unwanted crop plants).

As used herein “Beta vulgaris growing areas” refers to agricultural areas where Beta vulgaris plants are cultivated (i.e. deliberately planted or sown), with the aim of harvesting, such as beet root harvesting or seed harvesting.

The methods and uses according to the invention as described herein, in certain aspects may be for increasing the yield of Beta vulgaris plants or plant parts (i.e. the cultivated Beta vulgaris plants, as opposed to for instance the weed beets). An increased yield may for instance be an increased amount of (cultivated) Beta vulgaris or an increased biomass of (cultivated) Beta vulgaris, such as increase amount of biomass of harvested or harvestable plant parts, such as the beet root. An increased yield may also be for instance in the case of sugar beets an overall increase sugar amount or content (e.g. an increased sugar yield per hectare).

As used herein, the term “growing season” generally refers to the time period between planting or sowing the Beta vulgaris plants or seeds and harvesting the Beta vulgaris plants, in particular the beet roots. Usually, the growing season is from April to October/November. The skilled person will understand however, that the growing season may be longer or shorter depending on for instance climate or weather conditions or geological conditions. It will be further understood that the growing season may shift, such as for instance in the production of winter beets or spring beets.

In certain aspects, the herbicides as described herein are applied in the methods and uses according to the invention as described herein at a dosage sufficient for controlling (e.g. killing, inhibiting growth, preventing or delaying flowering, etc.) unwanted vegetation, such as bolters, weed beets, or annual beets. In certain embodiments, such dosage is as recommended by the manufacturer. This dosage preferably refers to a single application dose. It will be understood that more than one application may be needed during the growing season, such as two applications or three applications. The dose of such subsequent applications may be the same or may be different than the dose of the first application.

In certain embodiments, the plants or plant parts according to the invention comprise one or more mutation in ALS in addition to the D371 mutation. In certain embodiments, the plants or plant parts according to the invention comprise one or more mutation in ALS in the alternative to the D371 mutation.

In certain embodiments, the plant or plant part comprises an ALS having one or more mutation selected from: at position 113 an amino acid different than alanine (A), at position 188 an amino acid different than proline (P), at position 196 an amino acid different than alanine (A), at position 372 an amino acid different than arginine (R), at position 569 an amino acid different than tryptophan (W), at position 648 an amino acid different than serine (S), at position 649 an amino acid different than glycine (G). In certain embodiments, the plant or plant part comprises a polynucleic acid encoding a mutated ALS having one or more mutation selected from: at position 113 an amino acid different than alanine (A), at position 188 an amino acid different than proline (P), at position 196 an amino acid different than alanine (A), at position 372 an amino acid different than arginine (R), at position 569 an amino acid different than tryptophan (W), at position 648 an amino acid different than serine (S), at position 649 an amino acid different than glycine (G). In certain embodiments, the plant or plant part comprises an endogenous ALS allele encoding an ALS protein having one or more mutation selected from: at position 113 an amino acid different than alanine (A), at position 188 an amino acid different than proline (P), at position 196 an amino acid different than alanine (A), at position 372 an amino acid different than arginine (R), at position 569 an amino acid different than tryptophan (W), at position 648 an amino acid different than serine (S), at position 649 an amino acid different than glycine (G). Amino acid substitutions are as defined herein elsewhere. In particular embodiments, the mutations are conservative amino acid substitutions.

In certain embodiments, the plants or plant parts according to the invention comprise an ALS protein (or a polynucleic acid encoding a (endogenous) ALS protein or an endogenous ALS allele encoding an ALS protein) having an amino acid at position 569 which is different than tryptophan, such as alanine, glycine, isoleucine, leucine, methionine, phenylalanine, proline, valine or arginine, preferably a leucine.

In certain embodiments, the plants or plant parts according to the invention comprise an ALS protein (or a polynucleic acid encoding a (endogenous) ALS protein or an endogenous ALS allele encoding an ALS protein) having an amino acid at position 371 which is different than aspartic acid, preferably a glutamic acid, and an amino acid at position 569 which is different than tryptophan, preferably a leucine.

These additional mutations may reside on the same allele or on a different allele (i.e. a double mutant ALS or two separate single mutant ALS).

In certain embodiments, the plants or plant parts according to the invention comprise one or more mutation in other genes than ALS, in particular mutations in other genes conferring herbicide resistance.

Glyphosate is a unique herbicide, because it is the only herbicide known to inhibit synthesis of the aromatic amino acids phenylalanine, tyrosine, and tryptophan. Plants that cannot synthesize these three amino acids are not vital. The affected enzyme of the biosynthetic pathway leading towards aromatic amino acids is 5-enolpyruvylshikimate-3-phosphate synthase (EPSPS), which catalyzes the reaction of shikimate-3-phosphate (S3P) and phosphoenolpyruvate (PEP) to form 5-enolpyruvylshikimate-3-phosphate (EPSP). Glyphosate shares structural similarities to PEP, binds to EPSPS and inhibits the enzyme's reaction in a competitive manner. Glyphosate is the only known herbicide acting on EPSPS. Inhibition of synthesis of aromatic amino acids causes more or less immediate stop of growth and eventually kills plants within days after application. Therefore, glyphosate is generally a non-selective herbicide and will severely injure or kill any living plant tissue that it comes in contact with. However, it can be used selectively in glyphosate-resistant crops, including sugar beet, corn, soybean, cotton, and canola.

In certain embodiments, the wild type Beta vulgaris epsp synthase has an amino acid sequence as provided in NCBI reference sequence XP_010692222.1. In certain embodiments, the wild type or native Beta vulgaris epsp synthase has an amino acid sequence having at least 90%, preferably at least 95%, more preferably at least 98%, such as at least 99% sequence identity, preferably over the entire length, to the sequence of NCBI reference sequence XP_010692222.1, and preferably has epsp synthase activity, with the proviso that amino acid residue at position 179 is proline, and optionally that the amino acid residue at position 175 is threonine.

In certain embodiments, the wild type Beta vulgaris epsp synthase gene has a sequence encoding an amino acid sequence as provided in NCBI reference sequence XP_010692222.1. In certain embodiments, the wild type or native Beta vulgaris epsp synthase gene has a sequence encoding an amino acid sequence having at least 90%, preferably at least 95%, more preferably at least 98%, such as at least 99% sequence identity, preferably over the entire length, to the sequence of NCBI reference sequence XP_010692222.1, and preferably has epsp synthase activity, with the proviso that amino acid residue at position 179 is proline, and optionally that the amino acid residue at position 175 is threonine.

Preferably, as used herein, where amino acid residue positions are referred to for the epsp synthase, the numbering corresponds to the amino acid positions in reference sequence XP_010692222.1. In a preferred embodiment, a mutated EPSPS comprises a mutation at amino acid position 179, in particular, position 179 is not proline, such as a P179S mutation. In a preferred embodiment, a mutated EPSPS comprises a mutation at amino acid position 175, in particular, position 175 is not threonine, such as a T1751 mutation. In certain embodiments, both mutations are present in EPSPS. Both mutations confer glyphosate resistance.

In certain embodiments, the plant or plant part of the present invention is preferably non-transgenic with regard to the ALS gene, which is endogenous (apart from comprising a mutation at amino acid position 371, or any of the other amino acid positions referred to herein). Of course, the present invention does not exclude that other foreign genes can be transferred to the plant either by genetic engineering or by conventional methods such as crossing. Said genes can be genes conferring herbicide tolerances, preferably conferring herbicide tolerances different from ALS inhibitor herbicide tolerances, genes improving yield, genes improving resistances to biological organisms, and/or genes concerning content modifications.

The term “transgenic” here means genetically modified by the introduction of a non-endogenous nucleic acid sequence. Typically a species-specific nucleic acid sequence is introduced in a form, arrangement or quantity into the cell in a location where the nucleic acid sequence does not occur naturally in the cell. While the Beta vulgaris plants according to the invention are preferably non-transgenic with respect to the mutated ALS synthase, it will be understood that such Beta vulgaris plants may be transgenic for other traits.

In an aspect, the invention relates to an (isolated) polynucleic acid encoding a mutated ALS protein as described herein elsewhere. In certain embodiments, the (isolated) polynucleic acid encodes an ALS protein which is at least 80% identical, preferably over its entire length, preferably at least 90% identical, more preferably at least 95% identical, such as at least 98% identical to an ALS protein having a sequence as set forth in SEQ ID NO: 3, with the proviso that the amino acid at position 371 is not aspartic acid. In certain embodiments, the (isolated) polynucleic acid is at least 80% identical, preferably over its entire length, preferably at least 90% identical, more preferably at least 95% identical, such as at least 98% identical with a sequence as set forth in SEQ ID NO: 1 (i.e. mutated ALS gene), with the proviso that the codon corresponding the ALS amino acid at position 371 does not encode aspartic acid. In certain embodiments, the (isolated) polynucleic acid is at least 80% identical, preferably over its entire length, preferably at least 90% identical, more preferably at least 95% identical, such as at least 98% identical with a sequence as set forth in SEQ ID NO: 2 (i.e. mutated ALS cDNA or coding sequence), with the proviso that the codon corresponding the ALS amino acid at position 371 does not encode aspartic acid. Preferably the ALS protein has ALS (enzymatic) activity, as defined herein elsewhere. In certain embodiments, the amino acid at position 371 is glutamic acid. In certain embodiments, the ALS protein has a sequence as set forth in SEQ ID NO: 3. In an aspect, the invention relates to a Beta vulgaris plant or part thereof, comprising such polynucleic acid. In a preferred embodiment, the Beta vulgaris plant or part thereof comprises such polynucleic acid at its endogenous locus, preferably under control of its endogenous promoter. Accordingly, in certain embodiments, the invention relates to a Beta vulgaris plant or part thereof in which such polynucleic acid is operatively linked to the native ALS promoter. It will be understood that the polynucleic acid may correspond to an ALS cDNA or may correspond to an ALS gene sequence.

In certain embodiments, the nucleic acid molecule as described herein comprises less than 50000 nucleotides. In certain embodiments, the nucleic acid molecule as described herein comprises less than 40000 nucleotides. In certain embodiments, the nucleic acid molecule as described herein comprises less than 30000 nucleotides. In certain embodiments, the nucleic acid molecule as described herein comprises less than 25000 nucleotides. In certain embodiments, the nucleic acid molecule as described herein comprises less than 20000 nucleotides. In certain embodiments, the nucleic acid molecule as described herein comprises less than 15000 nucleotides. In certain embodiments, the nucleic acid molecule as described herein comprises less than 10000 nucleotides. In certain embodiments, the nucleic acid molecule as described herein comprises less than 5000 nucleotides. In certain embodiments, the nucleotide molecule as described herein comprises at least 100 nucleotides. In certain embodiments, the nucleic acid molecule as described herein comprises at least 100 nucleotides and less than 50000 nucleotides. In certain embodiments, the nucleic acid molecule as described herein comprises at least 100 nucleotides and less than 40000 nucleotides. In certain embodiments, the nucleic acid molecule as described herein comprises at least 100 nucleotides and less than 30000 nucleotides. In certain embodiments, the nucleic acid molecule as described herein comprises at least 100 nucleotides and less than 25000 nucleotides. In certain embodiments, the nucleic acid molecule as described herein comprises at least 100 nucleotides and less than 20000 nucleotides. In certain embodiments, the nucleic acid molecule as described herein comprises at least 100 nucleotides and less than 15000 nucleotides. In certain embodiments, the nucleic acid molecule as described herein comprises at least 100 nucleotides and less than 10000 nucleotides. In certain embodiments, the nucleic acid molecule as described herein comprises at least 100 nucleotides and less than 5000 nucleotides.

In an aspect, the invention relates to a vector comprising a polynucleic acid as referred to herein. The vector can be any vector known in the art, such as a prokaryotic vector or a eukaryotic vector. In certain embodiments, the polynucleic acid is operatively linked to one or more regulatory sequence in the vector, such as a promoter, as is known in the art. In certain embodiments, the promoter is a plant promoter. In certain embodiments, the promoter is a constitutive promoter. In certain embodiments, the promoter is an inducible promoter.

As used herein, a “vector” has its ordinary meaning in the art, and may for instance be a plasmid, a cosmid, a phage or an expression vector, a transformation vector, shuttle vector, or cloning vector; it may be double- or single-stranded, linear or circular; or it may transform a prokaryotic or eukaryotic host, either via integration into its genome or extrachromosomally. The nucleic acid according to the invention is preferably operatively linked in a vector with one or more regulatory sequences which allow the transcription, and, optionally, the expression, in a prokaryotic or eukaryotic host cell. A regulatory sequence-preferably, DNA—may be homologous or heterologous to the nucleic acid according to the invention. For example, the nucleic acid is under the control of a suitable promoter or terminator. Suitable promoters may be promoters which are constitutively induced (example: 35S promoter from the “Cauliflower mosaic virus” (Odell et al., 1985); those promoters which are tissue-specific are especially suitable (example: Pollen-specific promoters, Chen et al. (2010), Zhao et al. (2006), or Twell et al. (1991)), or are development-specific (example: blossom-specific promoters). Suitable promoters may also be synthetic or chimeric promoters which do not occur in nature, are composed of multiple elements, and contain a minimal promoter, as well as—upstream of the minimum promoter—at least one cis-regulatory element which serves as a binding location for special transcription factors. Chimeric promoters may be designed according to the desired specifics and are induced or repressed via different factors. Examples of such promoters are found in Gurr & Rushton (2005) or Venter (2007). For example, a suitable terminator is the nos-terminator (Depicker et al., 1982). The vector may be introduced via conjugation, mobilization, biolistic transformation, agrobacteria-mediated transformation, transfection, transduction, vacuum infiltration, or electroporation.

The vector may be a plasmid, a cosmid, a phage or an expression vector, a transformation vector, shuttle vector, or cloning vector; it may be double- or single-stranded, linear or circular. The vector may transform a prokaryotic or eukaryotic host, either via integration into its genome or extrachromosomally.

In certain embodiments, the vector is an expression vector. The nucleic acid is preferably operatively linked in a vector with one or more regulatory sequences which allow the transcription, and optionally the expression, in a prokaryotic or eukaryotic host cell. A regulatory sequence may be homologous or heterologous to the nucleic acid. For example, the nucleic acid is under the control of a suitable promoter or terminator. Suitable promoters may be promoters which are constitutively induced, for example, the 35S promoter from the “Cauliflower mosaic virus” (Odell et al., 1985. Identification of DNA sequences required for activity of the cauliflower mosaic virus 35S promoter.) Tissue-specific promoters, e.g. pollen-specific promoters as described in Chen et al. (2010. Molecular Biology Reports 37(2):737-744), Zhao et al. (2006. Planta 224(2): 405-412), or Twell et al. (1991. Genes & Development 5(3): 496-507), are particularly suitable, as are development-specific promoters, e.g. blossom-specific promoters. Suitable promoters may also be synthetic or chimeric promoters which do not occur in nature, and which are composed of multiple elements. Such synthetic or chimeric promoter may contain a minimal promoter, as well as at least one cis-regulatory element which serves as a binding location for special transcription factors. Chimeric promoters may be designed according to the desired specifics and can be induced or repressed via different factors. Examples of such promoters are found in Gurr & Rushton (2005. Trends in Biotechnology 23(6): 275-282) or Venter (2007. Trends in Plant Science: 12(3): 118-124). For example, a suitable terminator is the nos-terminator (Depicker et al., 1982. Journal of Molecular and Applied Genetics 1(6): 561-573).

In certain embodiments, the vector is a conditional expression vector. In certain embodiments, the vector is a constitutive expression vector. In certain embodiments, the vector is a tissue-specific expression vector, such as a leaf-specific expression vector. In certain embodiments, the vector is an inducible expression vector. All such vectors are well-known in the art.

Methods for preparation of the described vectors are commonplace to the person skilled in the art (Sambrook et al., 2001).

Also envisaged herein is a host cell, such as a plant cell or a (plant) protoplast, which comprises a nucleic acid as described herein, or a vector as described herein. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

The host cell may be a prokaryotic (for example, bacterial) or eukaryotic cell (for example, a plant cell or a yeast cell). For example, the host cell may be an Agrobacterium, such as Agrobacterium tumefaciens or Agrobacterium rhizogenes. Preferably, the host cell is a plant cell.

A nucleic acid described herein or a vector described herein may be introduced in a host cell via well-known methods, which may depend on the selected host cell, including, for example, conjugation, mobilization, biolistic transformation, agrobacteria-mediated transformation, transfection, transduction, vacuum infiltration, or electroporation. In particular, methods for introducing a nucleic acid or a vector in an Agrobacterium cell are well-known to the skilled person and may include conjugation or electroporation methods. Also methods for introducing a nucleic acid or a vector into a plant cell are known (Sambrook et al., 2001) and may include diverse transformation methods such as biolistic transformation and Agrobacterium-mediated transformation.

In particular embodiments, the present invention relates to a transgenic plant cell which comprises a nucleic acid as described herein, in particular an induction-promoting nucleic acid or a nucleic acid encoding a double-stranded RNA as described herein, as a transgene or a vector as described herein. In further embodiments, the present invention relates to a transgenic plant or a part thereof which comprises the transgenic plant cell.

For example, such a transgenic plant cell or transgenic plant is a plant cell or plant which is, preferably stably, transformed with a nucleic acid as described herein or a vector as described herein.

Preferably, the nucleic acid in the transgenic plant cell is operatively linked with one or more regulatory sequences which allow the transcription, and optionally the expression, in the plant cell. A regulatory sequence may be homologous or heterologous to the nucleic acid. The total structure made up of the nucleic acid according to the invention and the regulatory sequence(s) may then represent the transgene.

In an aspect, the invention relates to a polynucleic acid, preferably an isolated polynucleic acid, capable of specifically hybridizing with any of the polynucleic acid molecules of the invention as described herein, or the complement or reverse complement thereof. In certain embodiments, such polynucleic acid is capable of specifically hybridizing with a nucleotide sequence molecule of SEQ ID NO: 1 or 2; or the complement or the reverse complement thereof. It will be understood that such polynucleic acid hybridizes specifically with the recited sequences if it does not (functionally) hybridize with related sequences (e.g. mutated genes versus wild type genes). Such polynucleic acids can therefore be used for instance to discriminate between the mutated ALS according to the invention and for instance wild type ALS. In certain embodiments, the polynucleic acid comprises less than 500 nucleotides, such as less than 400 nucleotides, such as less than 300 nucleotides, such as less than 200 nucleotides, such as less than 100, nucleotides, such as preferably less than 80 nucleotides, more preferably less than 60 nucleotides, most preferably less than 50 nucleotides. In certain embodiments, such polynucleic acids comprise at least 5 nucleotides, preferably at least 10 nucleotides, more preferably at least 15 nucleotides. In certain embodiments, such polynucleic acid comprises 5 to 500 nucleotides, preferably 10 to 100 nucleotides, more preferably 15 to 50 nucleotides, such as 20 to 50 nucleotides. In certain embodiments, such polynucleic acids are primers or probes, as described herein elsewhere, such as a KASP primer (kompetitive allele specific PCR).

In certain embodiments, such polynucleic acid comprises at least the 10 most 3′ nucleotides of SEQ ID NO: 7, preferably at least the 15 most 3′ nucleotides, such as at least the 20 most 3′ nucleotides, the complement thereof, or the reverse complement thereof. In certain embodiments, such polynucleic acid comprises or consists of a sequence as set forth in SEQ ID NO: 7, the complement thereof, or the reverse complement thereof.

In an aspect, the invention relates to the use of the polynucleic acids (in particular polynucleic acids encoding mutant ALS), vector, or host cells as described herein for generating the Beta vulgaris plants or plant parts according to the invention as described herein elsewhere, in particular a Beta vulgaris plant or plant part comprising an (endogenous) ALS protein having at position 371 an amino acid different than aspartic acid.

In an aspect, the invention relates to the use of the polynucleic acids, in particular primers or probes, as described herein for identifying the Beta vulgaris plants or plant parts according to the invention as described herein elsewhere, in particular a Beta vulgaris plant or plant part comprising an (endogenous) ALS protein having at position 371 an amino acid different than aspartic acid.

In an aspect, the invention relates to a Beta vulgaris plant of plant part comprising:
a) a polynucleic acid comprising a sequence as set forth in SEQ ID NO: 1;
b) a polynucleic acid comprising a sequence as set forth in SEQ ID NO: 2;
c) a polynucleic acid encoding an ALS protein having a cDNA sequence as set forth in SEQ ID NO: 2;
d) a polynucleic acid encoding an ALS protein having a sequence as set forth in SEQ ID NO: 3.

In certain embodiments, such Beta vulgaris plant or plant part further comprises:
a) a polynucleic acid comprising a sequence as set forth in SEQ ID NO: 10;
b) a polynucleic acid comprising a sequence as set forth in SEQ ID NO: 11;
c) a polynucleic acid encoding an ALS protein having a cDNA sequence as set forth in SEQ ID NO: 11;
d) a polynucleic acid encoding an ALS protein having a sequence as set forth in SEQ ID NO: 12.

It will be understood that preferably these polynucleic acids are comprised in the genome of the plant or plant part. Preferably, these polynucleic acids are conditionally or constitutively expressed, and hence are under control of suitable regulatory sequences, as described herein elsewhere. In a preferred embodiment, these polynucleic acids are at the endogenous ALS location under the control of the endogenous ALS promoter.

In an aspect, the invention relates to a method for generating a Beta vulgaris plant or plant part, such as a Beta vulgaris plant or plant part according to the invention as described herein, comprising introducing in a plant or a plant part, such as a protoplast, a polynucleic acid according to the invention, preferably in the genome of the plant or plant part, in particular a polynucleic acid encoding a (endogenous) mutated ALS according to the invention as defined herein elsewhere. In certain embodiments, such method further comprises regenerating a plant from a plant part, such as a protoplast.

In an aspect, the invention relates to a method for generating a Beta vulgaris plant or plant part, such as a Beta vulgaris plant or plant part according to the invention as described herein, comprising mutating in a plant or a plant part, such as a protoplast or seed, a polynucleic acid encoding a (endogenous) ALS, preferably in the genome of the plant or plant part, in particular a polynucleic acid encoding a (endogenous) mutated ALS according to the invention as defined herein elsewhere. In certain embodiments, such method further comprises regenerating a plant from a plant part, such as a protoplast or seed.

Mutagenesis may be performed in accordance with any of the techniques known in the art. As used herein, “mutagenization” or “mutagenesis” includes both conventional mutagenesis and location-specific mutagenesis or “genome editing” or “gene editing”. In conventional mutagenesis, modification at the DNA level is not produced in a targeted manner. The plant cell or the plant is exposed to mutagenic conditions, such as TILLING, via UV light exposure or the use of chemical substances (Till et al., 2004). An additional method of random mutagenesis is mutagenesis with the aid of a transposon. Location-specific mutagenesis enables the introduction of modification at the DNA level in a target-oriented manner at predefined locations in the DNA. For example, TALENS, meganucleases, homing endonucleases, zinc finger nucleases, or a CRISPR/Cas System may be used for this.

In certain embodiments, the nucleic acid modification of the ALS gene is effected by random mutagenesis. Cells or organisms may be exposed to mutagens such as UV radiation or mutagenic chemicals (such as for instance such as ethyl methanesulfonate (EMS)), and mutants with desired characteristics are then selected. Mutants can for instance be identified by TILLING (Targeting Induced Local Lesions in Genomes). The method combines mutagenesis, such as mutagenesis using a chemical mutagen such as ethyl methanesulfonate (EMS) with a sensitive DNA screening-technique that identifies single base mutations/point mutations in a target gene. The TILLING method relies on the formation of DNA heteroduplexes that are formed when multiple alleles are amplified by PCR and are then heated and slowly cooled. A “bubble” forms at the mismatch of the two DNA strands, which is then cleaved by a single stranded nucleases. The products are then separated by size, such as by HPLC. See also McCallum et al. “Targeted screening for induced mutations”; Nat Biotechnol. 2000 April; 18(4):455-7 and McCallum et al. “Targeting induced local lesions IN genomes (TILLING) for plant functional genomics”; Plant Physiol. 2000 June; 123(2):439-42.

In certain embodiments, the mutant ALS can be obtained by targeted mutagenesis, such as gene editing techniques, including CRISPR/Cas, zinc finger nucleases, meganucleases, or TALEN gene editing techniques, as are known in the art.

In certain embodiments, mutated ALS according to the invention can be obtained by:
a) mutagenizing Beta vulgaris cells or tissue, such as seeds, with EMS and/or EMU, such as at least 0.5% EMS or at least 0.3% ENU,
b) producing stecks from the mutagenized cells or tissue (M0),
c) replanting stecks for producing a population of seeds (M1),
d) producing seeds (M2) from plants grown from M1 seeds,
e) sowing M2 seeds and applying an ALS inhibitor herbicide,
f) optionally, replanting surviving plants in pots and applying an ALS inhibitor herbicide to the growing plants, and
g) selecting surviving plants without herbicide damages and/or selecting plants comprising an ALS allele encoding an ALS protein comprising at position 371 an amino acid different than aspartate (D).

The mutant ALS according to the invention can also be obtained by introgression.

As used herein, the terms “introgression”, “introgressed” and “introgressing” refer to both a natural and artificial process whereby chromosomal fragments or genes of one plant, species, variety or cultivar are moved into the genome of another plant, species, variety or cultivar, by crossing those plants or species. The process may optionally be completed by backcrossing to the recurrent parent. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like or may be identified for instance through standard techniques such as sequencing, hybridization, PCR, etc, as defined herein elsewhere. In any case, offspring comprising the desired allele can be repeatedly backcrossed to a line having a desired genetic background and selected for the desired allele, to result in the allele becoming fixed in a selected genetic background. The process of “introgressing” is often referred to as “backcrossing” when the process is repeated two or more times. “Introgression fragment” or “introgression segment” or “introgression region” refers to a chromosome fragment (or chromosome part or region) which has been introduced into another plant of the same or related species either artificially or naturally such as by crossing or traditional breeding techniques, such as backcrossing, i.e. the introgressed fragment is the result of breeding methods referred to by the verb “to introgress” (such as backcrossing). It is understood that the term “introgression fragment” never includes a whole chromosome, but only a part of a chromosome. The introgression fragment can be large, e.g. even three quarter or half of a chromosome, but is preferably smaller, such as about 15 Mb or less, such as about 10 Mb or less, about 9 Mb or less, about 8 Mb or less, about 7 Mb or less, about 6 Mb or less, about 5 Mb or less, about 4 Mb or less, about 3 Mb or less, about 2.5 Mb or 2 Mb or less, about 1 Mb (equals 1,000,000 base pairs) or less, or about 0.5 Mb (equals 500,000 base pairs) or less, such as about 200,000 bp (equals 200 kilo base pairs) or less, about 100,000 bp (100 kb) or less, about 50,000 bp (50 kb) or less, about 25,000 bp (25 kb) or less. In certain embodiments, the introgression fragment comprises, consists of, or consists essentially of the mutated ALS (allele) according to the invention as described herein.

A genetic element, an introgression fragment, or a gene or allele conferring a trait (such as ALS inhibitor herbicide tolerance) is said to be “obtainable from” or can be “obtained from” or “derivable from” or can be “derived from” or “as present in” or “as found in” a plant or plant part as described herein elsewhere if it can be transferred from the plant in which it is present into another plant in which it is not present (such as a line or variety) using traditional breeding techniques without resulting in a phenotypic change of the recipient plant apart from the addition of the trait conferred by the genetic element, locus, introgression fragment, gene or allele. The terms are used interchangeably and the genetic element, locus, introgression fragment, gene or allele can thus be transferred into any other genetic background lacking the trait. Not only pants comprising the genetic element, locus, introgression fragment, gene or allele can be used, but also progeny/descendants from such plants which have been selected to retain the genetic element, locus, introgression fragment, gene or allele, can be used and are encompassed herein. Whether a plant (or genomic DNA, cell or tissue of a plant) comprises the same genetic element, locus, introgression fragment, gene or allele as obtainable from such plant can be determined by the skilled person using one or more techniques known in the art, such as phenotypic assays, whole genome sequencing, molecular marker analysis, trait mapping, chromosome painting, allelism tests and the like, or combinations of techniques. It will be understood that transgenic plants may also be encompassed.

As used herein the terms “genetic engineering”, “transformation” and “genetic modification” are all used herein as synonyms for the transfer of isolated and cloned genes into the DNA, usually the chromosomal DNA or genome, of another organism.

“Transgenic” or “genetically modified organisms” (GMOs) as used herein are organisms whose genetic material has been altered using techniques generally known as “recombinant DNA technology”. Recombinant DNA technology encompasses the ability to combine DNA molecules from different sources into one molecule ex vivo (e.g. in a test tube). This terminology generally does not cover organisms whose genetic composition has been altered by conventional cross-breeding or by “mutagenesis” breeding, as these methods predate the discovery of recombinant DNA techniques. “Non-transgenic” as used herein refers to plants and food products derived from plants that are not “transgenic” or “genetically modified organisms” as defined above.

“Transgene” or “chimeric gene” refers to a genetic locus comprising a DNA sequence, such as a recombinant gene, which has been introduced into the genome of a plant by transformation, such as Agrobacterium mediated transformation. A plant comprising a transgene stably integrated into its genome is referred to as “transgenic plant”.

“Gene editing” or “genome editing” refers to genetic engineering in which in which DNA or RNA is inserted, deleted, modified or replaced in the genome of a living organism. Gene editing may comprise targeted or non-targeted (random) mutagenesis. Targeted mutagenesis may be accomplished for instance with designer nucleases, such as for instance with meganucleases, zinc finger nucleases (ZFNs), transcription activator-like effector-based nucleases (TALEN), and the clustered regularly interspaced short palindromic repeats (CRISPR/Cas9) system. These nucleases create site-specific double-strand breaks (DSBs) at desired locations in the genome. The induced double-strand breaks are repaired through nonhomologous end-joining (NHEJ) or homologous recombination (HR), resulting in targeted mutations or nucleic acid modifications. The use of designer nucleases is particularly suitable for generating gene knockouts or knockdowns. In certain embodiments, designer nucleases are developed which specifically induce a mutation in the ALS gene, as described herein elsewhere. Delivery and expression systems of designer nuclease systems are well known in the art.

In certain embodiments, the nuclease or targeted/site-specific/homing nuclease is, comprises, consists essentially of, or consists of a (modified) CRISPR/Cas system or complex, a (modified) Cas protein, a (modified) zinc finger, a (modified) zinc finger nuclease (ZFN), a (modified) transcription factor-like effector (TALE), a (modified) transcription factor-like effector nuclease (TALEN), or a (modified) meganuclease. In certain embodiments, said (modified) nuclease or targeted/site-specific/homing nuclease is, comprises, consists essentially of, or consists of a (modified) RNA-guided nuclease. It will be understood that in certain embodiments, the nucleases may be codon optimized for expression in plants. As used herein, the term “targeting” of a selected nucleic acid sequence means that a nuclease or nuclease complex is acting in a nucleotide sequence specific manner. For instance, in the context of the CRISPR/Cas system, the guide RNA is capable of hybridizing with a selected nucleic acid sequence.

Gene editing may involve transient, inducible, or constitutive expression of the gene editing components or systems. Gene editing may involve genomic integration or episomal presence of the gene editing components or systems. Gene editing components or systems may be provided on vectors, such as plasmids, which may be delivered by appropriate delivery vehicles, as is known in the art. Preferred vectors are expression vectors.

Gene editing may comprise the provision of recombination templates, to effect homology directed repair (HDR). For instance a genetic element may be replaced by gene editing in which a recombination template is provided. The DNA may be cut upstream and downstream of a sequence which needs to be replaced. As such, the sequence to be replaced is excised from the DNA. Through HDR, the excised sequence is then replaced by the template. In certain embodiments, the mutated ALS gene or cDNA of the invention as described herein (or a fragment thereof comprising the mutation of the invention, i.e. corresponding to an amino acid which is different from aspartic acid at amino acid position 371 of ALS) may be provided on/as a template. By designing the system such that double strand breaks are introduced upstream and downstream of the corresponding region in the genome of a plant not comprising the mutant ALS, this region is excised and can be replaced with the template comprising the mutant ALS (or fragment) of the invention. In this way, introduction of the mutant ALS of the invention in a plant need not involve multiple backcrossing, in particular in a plant of specific genetic background.

In an aspect, the invention relates to a Beta vulgaris plant (or plant part) which is (directly) obtained by or which is obtainable from the methods for generating a Beta vulgaris plant according to the invention as described herein. In a preferred embodiment, the plant part is a root beet (or beet root).

In certain embodiments, the mutant ALS in the Beta vulgaris plant or plant part is homozygous. In certain embodiments, the mutant ALS in the Beta vulgaris plant or plant part is heterozygous, i.e. the plant or plant part comprises one mutant ALS allele and one wild type allele, or alternatively one mutant ALS allele according to the invention (i.e. having at amino acid position 371 an amino acid which is different than aspartic acid, as described herein elsewhere), and one mutant ALS allele having a different mutation (such as preferably having at amino acid position 569 an amino acid which is different than tryptophan, as described herein elsewhere).

In certain embodiments, the Beta vulgaris plant or plant part of the invention comprises an ALS protein having at amino acid position 371 an amino acid which is different from aspartic acid as described herein elsewhere and at amino acid position 569 an amino acid which is different from tryptophan as described herein elsewhere (i.e. a double mutation in the same allele).

In an aspect, the invention relates to a method for identifying a Beta vulgaris plant or plant part, such as a Beta vulgaris plant or plant part according to the invention as described herein, comprising screening in an ALS protein for the presence of an amino acid at position 371 which is different than aspartate (D), or screening for the presence of a codon encoding an amino acid in an ALS protein at position 371 which is different than aspartate (D). The method may further comprise the step of selecting a Beta vulgaris plant or plant part if an amino acid at position 371 in an ALS protein which is different than aspartate (D) is identified or if a codon encoding an amino acid in an ALS protein at position 371 which is different than aspartate (D) is identified.

In an aspect, the invention relates to a method for identifying a Beta vulgaris plant or plant part, such as a Beta vulgaris plant or plant part which is tolerant to or which has increased tolerance to one or more ALS inhibitor herbicide, comprising screening in an ALS protein of a Beta vulgaris plant for the presence of an amino acid at position 371 which is different than aspartate (D), or screening for the presence of a codon encoding an amino acid in an ALS protein at position 371 which is different than aspartate (D). The method may further comprise the step of identifying a Beta vulgaris plant or plant part which is tolerant to or has increased tolerance to one or more ALS inhibitor herbicide if an amino acid at position 371 in an ALS protein which is different than aspartate (D) is identified or if a codon encoding an amino acid in an ALS protein at position 371 which is different than aspartate (D) is identified. The method may further comprise the step of selecting a Beta vulgaris plant or plant part which is tolerant to or has increased tolerance to one or more ALS inhibitor herbicide if an amino acid at position 371 in an ALS protein which is different than aspartate (D) is identified or if a codon encoding an amino acid in an ALS protein at position 371 which is different than aspartate (D) is identified.

In an aspect, the invention relates to a method for detecting or identifying the ALS mutations according to the invention as described herein.

Any means of detection can be applied, as described herein elsewhere, and include for instance sequencing, hybridization based methods (such as (dynamic) allele-specific hybridization, molecular beacons, SNP microarrays), enzyme based methods (such as PCR, KASP (Kompetitive Allele Specific PCR), RFLP, ALFP, RAPD, Flap endonuclease, primer extension, 5′-nuclease, oligonucleotide ligation assay), post-amplification methods based on physical properties of DNA (such as single strand conformation polymorphism, temperature gradient gel electrophoresis, denaturing high performance liquid chromatography, high-resolution melting of the entire amplicon, use of DNA mismatch-binding proteins, SNPlex, surveyor nuclease assay), etc.

In certain embodiments, detection of the mutant ALS is performed by KASP. In certain embodiments, an allele specific KASP primer (for the mutant ALS) comprises at least the 10 most 3′ nucleotides of SEQ ID NO: 7, preferably at least the 15 most 3′ nucleotides, such as at least the 20 most 3′ nucleotides, the complement thereof, or the reverse complement thereof. In certain embodiments, such KSAP primer comprises or consists of a sequence as set forth in SEQ ID NO: 7, the complement thereof, or the reverse complement thereof.

In certain embodiments, detection of the wild type ALS is performed by KASP. In certain embodiments, an allele specific KASP primer (for the wild type ALS) comprises at least the 10 most 3′ nucleotides of SEQ ID NO: 8, preferably at least the 15 most 3′ nucleotides, such as at least the 20 most 3′ nucleotides, the complement thereof, or the reverse complement thereof. In certain embodiments, such KSAP primer comprises or consists of a sequence as set forth in SEQ ID NO: 8, the complement thereof, or the reverse complement thereof.

The common KASP primer (which is used in the detection of both the mutant and wild type ALS) can be appropriately chosen by the skilled person, and is not particularly limited in location. In certain embodiments, the common primer comprises or consists of a sequence as set forth in SEQ ID NO: 9.

In an aspect, the invention relates to a kit comprising a polynucleic acid, in particular a primer or probe as described herein, and optionally reagents for detecting a mutant ALS or for discriminating between a mutant ALS and a wild type ALS, such as the KASP primers described herein.

In an aspect, the invention relates to a method for producing Beta vulgaris root beets (or beet roots), comprising sowing or plants Beta vulgaris plant according to the invention as described herein elsewhere, and harvesting root beets (or beet roots), preferably at the end of the growing season.

In an aspect, the invention relates to the use of a Beta vulgaris plant or plant part, preferably a root beet (or beet root) in a method for sugar production, anaerobic digestion, or fermentation.

In an aspect, the invention relates to the use of a Beta vulgaris plant or plant part, preferably a root beet (or beet root) in a method for biogas or biofuel production.

“Fermentation” as used herein refers to the process of transforming an organic molecule into another molecule using a micro-organism. For example, “fermentation” can refer to aerobic transforming sugars or other molecules from plant material, such as the plant material of the present invention, to produce alcohols (e.g., ethanol, methanol, butanol); organic acids [e.g., citric acid, acetic acid, itaconic acid, lactic acid, gluconic acid); ketones [e.g., acetone), amino acids {e.g., glutamic acid); gases {e.g., H2 and CO2), antibiotics {e.g., penicillin and tetracycline); enzymes; vitamins {e.g., riboflavin, B12, beta-carotene); and/or hormones. Fermentation include fermentations used in the consumable alcohol industry {e.g., beer and wine). Fermentation also includes anaerobic fermentations, for example, for the production of biofuels. Fermenting can be accomplished by any organism suitable for use in a desired fermentation step, including, but not limited to, bacteria, fungi, archaea, and protists. Suitable fermenting organisms include those that can convert mono-, di-, and tri-saccharides, especially glucose and maltose, or any other biomass-derived molecule, directly or indirectly to the desired fermentation product (e.g., ethanol, butanol, etc.). Suitable fermenting organisms also include those which can convert non-sugar molecules to desired fermentation products. Such organisms and fermentation methods are known to the person skilled in the art.

The term “biofuel”, as used herein, refers to a fuel that is derived from biomass, i.e., a living or recently living biological organism, such as a plant or an animal waste. Biofuels include, but are not limited to, biodiesel, biohydrogen, biogas, biomass-derived dimethylfuran (DMF), and the like. In particular, the term “biofuel” can be used to refer to plant-derived alcohols, such as ethanol, methanol, propanol, or butanol, which can be denatured, if desired prior to use. The term “biofuel” can also be used to refer to fuel mixtures comprising plant-derived fuels, such as alcohol/gasoline mixtures (i.e., gasohols). Gasohols can comprise any desired percentage of plant-derived alcohol (i.e., about 10%, 15%, 20%, 25%, 30%, 35%, 40%, 45%, 50%, 55%, 60%, 65%, 70%, 75%, 80%, 85%, 90%, or 95% plant-derived alcohol). For example, one useful biofuel-based mixture is E85, which comprises 85% ethanol and 15% gasoline. The biofuel can be any biofuel produced by aerobic or anaerobic fermentation of plant material. A non-limiting example of a biofuel obtained by aerobic fermentation is bioethanol. Biofuels that can be obtained by anaerobic fermentation include, but are not limited to biogas and/or biodiesel. Methods of aerobic and/or anaerobic fermentation are known to the person skilled in the art. Further encompassed by the present invention are biofuels selected from the group comprising ethanol, biogas and/or biodiesel as produced by the method for producing one or more biofuel(s) or the present invention.

The present invention includes other industrial applications such as the production of antibodies or bioplastic in the sugar beet plants of the present invention. Further, sugar beet plants of the present invention or parts thereof can be also be used without further processing, such as, for example, as cattle feed.

The term “sugar” refers to fermentable monosaccharides disaccharides, and trisaccharides, particularly to mono- and disaccharides. Thus, in the present invention sugars include, but are not limited to, sucrose, fructose, glucose, galactose, maltose, lactose, and mannose, preferably sucrose.

The aspects and embodiments of the invention are further supported by the following non-limiting examples.

### EXAMPLES

### Example 1

250 kg of ethyl methanesulfonate (EMS) and N-ethyl-N-nitrosourea (ENU) mutagenized sugar beet seeds of KWS proprietary genotype of generation M2 were sown on a 15 ha field. Six weeks and eight weeks after emergence the field was sprayed with herbicide CONVISO® ONE (50 g/L Foramsulfuron, 30 g/L Thiencarbazon-methyl) at a concentration of 0.5 L ha-1. Two weeks after the second treatment surviving plants were selected, transplanted, and further cultivated in greenhouses. One more CONVISO® ONE (0.5 L ha-1) treatment was applied in the greenhouse. In total, seven plants were selected. DNA samples were extracted from all five individuals and BvALS coding sequence was sequenced using standard methods (Sanger sequencing).

Two mutants, named 18ZZJZJ7MS1001-0019 and 18ZZJZJ7MS1001-0023 happened to carry identical T to A mutations at position 1141 (of the BvALS_g8976.t1_T807_genomic_DNA reference sequence SEQ ID NO: 1 (position 1113 of the provided CDS, SEQ ID NO: 2) translating to an amino acid change from Asp (D) to Glu (E) at position 371 of the BvALS protein sequence (SEQ ID NO: 3). For the three other mutants sequencing was not clear and it was assumed first that they were not mutated within the BvALS protein coding sequence.

For determination of the zygosity state and to clearly identify the point mutation leading to substitution D371E in BvALS the specific marker sytxalss14as001 applicable as KASP marker has been developed:
Primer AlleleX (mutant allele):

(SEQ ID NO: 7)
GAAGGTGACCAAGTTCATGCTTTCGGGGTTAGGTTTGATGAA

Primer_AlleleY (wild type allele):

(SEQ ID NO: 8)
GAAGGTCGGAGTCAACGGATTGCTTTCGGGGTTAGGTTTGATGAT

Primer_Common:

(SEQ ID NO: 9)
GGCTAGCAAACGCCTCGAGCTT

Marker application showed that mutant plant 18ZZJZJ7MS1001-0019 and 18ZZJZJ7MS1001-0023 are homozygous for the point mutation leading to substitution D371E in BvALS. By means of the marker, two of the three unknown mutants, 18ZZJZJ7MS1001-0018 und 18ZZJZJ7MS1001-0030, have been identified as being heterozygous for the point mutation leading to substitution D371E in BvALS.

The identified ALS mutants have been tested repeatedly in CONVISO® ONE treatments in concentrations used for field treatments as well as in treatment with the individual active ingredients of CONVISO® ONE, Foramsulfuron and Thiencarbazon-methyl, treatment with Pulsar® 40 (active ingredient: Imazamox), treatment with active ingredient Bispyribac, and treatment with Broadway® (active ingredients: Florasulam, Pyroxylam) (Table 1). All herbicide treatments has been conducted with amount corresponding to 1× recommended field application concentration: Offspring seed of both, a plant homozygous and heterozygous for BvALS_D371E mutation as well as the M0-background genotype were sown, cultivated in a greenhouse and transplanted to single pots after emergence. Herbicide treatments were performed when first true leaves appeared.

TABLE 1
ALS inhibitor herbicide treatments

| | | Herbicide | Concentration | | Application | |
|---|---|---|---|---|---|---|
| | Compound | Name | g/l or g/kg | | Dose | |
| Compound Mix | Foramsulfuron | CONVISO ® | 50 | g/l | 1 | l/ha |
| | Thiencarbazon-methyl | ONE | 30 | g/l | | |
| Sulfonylureas | Foramsulfuron | pure | 50 | g/l | 1 | l/ha |
| | | compound | | | | |
| Sulfonylaminocarb | Thiencarbazon-methyl | pure | 30 | g/l | 1 | l/ha |
| onyltriazolinone | | compound | | | | |
| Imidazolinones | Imazamox | Pulsar ® 40 | 40 | g/l | 1.25 | l/ha |
| Pyrimidinylthio- | Bispyribac | pure | 100 | g/l | 1 | l/ha |
| benzates | | compound | | | | |
| Triazolopyrimidines | Florasulam | Broadway ® | 22.8 | g/kg | 0.275 | kg/ha |
| | Pyroxylam | | 68.3 | g/kg | | |

Tolerance ratings were taken 14 days after treatment. Growing of the first pair of true leaves was used as indicator for herbicide tolerance. Surprisingly, homozygous as well as heterozygous BvALS_D371E mutants survived four of the five known classes of ALS inhibitors, plus CONVISO® ONE as a mixture of two such classes without visible, phytotoxic herbicide damages (Table 2).

TABLE 2
Testing of different classes of ALS inhibitors

| | Homozygous mutants | Heterozygous mutants |
|---|---|---|
| Compound Mix | Tolerant | tolerant |
| Sulfonylureas | Tolerant | tolerant |
| Sulfonylaminocarb | Tolerant | tolerant |
| onyltriazolinone | | |
| Imidazolinones | Tolerant | tolerant |
| Pyrimidinylthio- | Tolerant | tolerant |
| benzates | | |
| Triazolopyrimidines | Sensitive | sensitive |

Further, one sugar beet genotype, homozygous for the mutation BvALS_D371E (RR) was crossed with a second sugar beet genotype. Progenies have been phenotypically analyzed with respect to tolerance towards CONVISO® ONE. Genetically a segregation in RR:Rs:ss (1:2:1) has been expected. The observation shows a phenotypic segregation in 3:1 (resistant:sensitive). This confirms again, that even heterozygous mutants exhibit a high level of tolerance suitable for commercial application.

1. A Beta vulgaris plant, plant part, or plant population comprising, expressing, or capable of expressing a mutated endogenous acetolactate synthase (ALS) protein or a polynucleic acid encoding a mutated endogenous acetolactate synthase (ALS) protein comprising at position 371 an amino acid different than aspartic acid (D).

2. The Beta vulgaris plant, plant part, or plant population according to claim 1, comprising a mutated endogenous allele encoding an ALS protein comprising at position 371 an amino acid different than aspartic acid (D).

3. The Beta vulgaris plant, plant part, or plant population according to claim 1 which is tolerant to one or more ALS inhibitor herbicide.

4. The Beta vulgaris plant, plant part, or plant population according to claim 1 which is tolerant to one or more ALS inhibitor herbicide selected from (sulfon)amides such as sulfonylureas, sulfonylaminocarbonyltriazolinones, sulfonanilides, or triazolopyrimidines; imidazolinones; and pyrimidinyl(thio/oxy)benzoates, preferably selected from sulfonylureas, sulfonylaminocarbonyltriazolinones, imidazolinones, and pyrimidinyl(thio/oxy)benzoates.

5. The Beta vulgaris plant, plant part, or plant population according to claim 1, comprising a polynucleic acid encoding a mutated endogenous acetolactate synthase (ALS) protein or comprising a mutated endogenous allele encoding an ALS protein comprising at position 569 an amino acid different than tryptophan (W).

6. The Beta vulgaris plant, plant part, or plant population according to claim 1, wherein said ALS is homozygous or heterozygous.

7. An isolated polynucleic acid encoding a mutated endogenous acetolactate synthase (ALS) protein as defined in claim 1.

8. The isolated polynucleic acid according to claim 7, wherein said ALS protein has a sequence which is at least 80% identical to SEQ ID NO: 3, preferably at least 90%, more preferably at least 95%, such as at least 98%.

9. A vector comprising the isolated polynucleic acid according to claim 7.

10. An isolated polynucleic acid specifically hybridizing with the polynucleic acid according to claim 7, the complement thereof, or the reverse complement thereof, preferably wherein said polynucleic acid is a primer or probe.

11. A method for identifying a Beta vulgaris plant or plant part, comprising screening for the presence in an ALS protein of an amino acid at position 371 which is different than aspartic acid (D), or screening for the presence of a codon encoding an amino acid in an ALS protein at position 371 which is different than aspartic acid (D).

12. A method for generating a Beta vulgaris plant or plant part, comprising mutating in the genome of a plant or plant part an endogenous ALS allele resulting in an ALS allele encoding an ALS protein comprising at position 371 an amino acid different than aspartic acid (D) or introducing in the genome (and expressing) of a plant or plant part a polynucleic acid encoding a mutated endogenous ALS protein comprising at position 371 an amino acid different than aspartic acid (D).

13. A Beta vulgaris plant or plant part directly obtained by or obtainable by the method according to claim 12, or the progeny thereof.

14. A method for controlling unwanted vegetation in Beta vulgaris growing areas or for increasing the yield in Beta vulgaris growing areas, comprising the steps of:
a) planting Beta vulgaris plants or sowing Beta vulgaris seeds according to claim 1,
b) applying one or more ALS inhibitor herbicide to the growing plants, preferably at a dosage sufficient for inhibiting the growth of the unwanted vegetation, more preferably at a dosage sufficient for killing the unwanted vegetation, and
c) optionally, repeating step b) during the growing season.

15. The method according to claim 14, wherein said unwanted vegetation comprises bolters, weed beets, or annual beets.


SEQUENCE LISTING (12 sequences)

SEQ ID NO: 1 - DNA, 2262 bp, synthetic construct
  misc_feature 1..2262: note = genomic DNA of mutant BvALS_D371E
  source 1..2262: mol_type = other DNA
tctctctccaaaactccaaacaacaacaatggcggctaccttcacaaacccaacattttccccttcctcaactcaattaaccaaaaccctaaaatcccaatcttccatttcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggtgcatctatggaaatccaccaagctctgacgcgctctaaaaccatccgcaatgtcctcccccgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccaattgttgaggtaacaaggtctattactaagcataattatttggttttggatgtagaagatattcctagaattgttaaggaagccttttttttagctaattctggcaggcctggacctgttttgattgatcttcctaaagatattcagcagcaactggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgaacgtgtgactgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaggtggagagaagaattaggtgagcagaagaaggaattcccactgagttttaagacatttggggatgcaatccctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaatgggaagataggttctataaagctaatcgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattgatcggtttaatgacggttggaaccatttaaagagggtaagctatattactgtatgtatattagtatgttcctggataatttagaagcttttgtctgttgtcttttgcagtttatgaagttagtttgctgttgtcatgttacttgttactttaaaaagctttttgtagtttttgagcaactagtatggaatgctcttcctgtattgcttggaaaattcacaaaagtggtttttcggct

SEQ ID NO: 2 - DNA, 1998 bp, synthetic construct
  misc_feature 1..1998: note = cDNA of mutant BvALS_D371E
  source 1..1998: mol_type = other DNA
atggcggctaccttcacaaacccaacattttccccttcctcaactcaattaaccaaaaccctaaaatcccaatcttccatttcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggtgcatctatggaaatccaccaagctctgacgcgctctaaaaccatccgcaatgtcctcccccgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccaattgttgaggtaacaaggtctattactaagcataattatttggttttggatgtagaagatattcctagaattgttaaggaagccttttttttagctaattctggcaggcctggacctgttttgattgatcttcctaaagatattcagcagcaactggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgaacgtgtgactgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaggtggagagaagaattaggtgagcagaagaaggaattcccactgagttttaagacatttggggatgcaatccctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaatgggaagataggttctataaagctaatcgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattga

SEQ ID NO: 3 - protein, 665 aa, synthetic construct
  REGION 1..665: note = protein of mutant BvALS_D371E
MAATFTNPTFSPSSTQLTKTLKSQSSISSTLPFSTPPKTPTPLFHRPLQISSSQSHKSSAIKTQTQAPSSPAIEDSSFVSRFGPDEPRKGSDVLVEALEREGVTNVFAYPGGASMEIHQALTRSKTIRNVLPRHEQGGVFAAEGYARATGKVGVCIATSGPGATNLVSGLADALLDSVPLVAITGQVPRRMIGTDAFQETPIVEVTRSITKHNYLVLDVEDIPRIVKEAFFLANSGRPGPVLIDLPKDIQQQLVVPDWDRPFKLGGYMSRLPKSKFSTNEVGLLEQIVRLMSESKKPVLYVGGGCLNSSEELRRFVELTGIPVASTLMGLGSYPCNDELSLHMLGMHGTVYANYAVDKADLLLAFGVRFDERVTGKLEAFASRAKIVHIDIDSAEIGKNKQPHVSICADVKLALRGMNKILESRIGKLNLDFSRWREELGEQKKEFPLSFKTFGDAIPPQYAIQVLDELTNGNAIISTGVGQHQMWAAQHYKYRNPRQWLTSGGLGAMGFGLPAAIGAAVARPDAVVVDIDGDGSFIMNVQELATIRVENLPVKIMLLNNQHLGMVVQWEDRFYKANRAHTYLGNPSKSADIFPDMLKFAEACDIPSARVSNVADLRAAIQTMLDTPGPYLLDVIVPHQEHVLPMIPSGAGFKDTITEGDGRTSY

SEQ ID NO: 4 - DNA, 2262 bp, Beta vulgaris
  source 1..2262: mol_type = genomic DNA
tctctctccaaaactccaaacaacaacaatggcggctaccttcacaaacccaacattttccccttcctcaactcaattaaccaaaaccctaaaatcccaatcttccatttcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggtgcatctatggaaatccaccaagctctgacgcgctctaaaaccatccgcaatgtcctcccccgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccaattgttgaggtaacaaggtctattactaagcataattatttggttttggatgtagaagatattcctagaattgttaaggaagccttttttttagctaattctggcaggcctggacctgttttgattgatcttcctaaagatattcagcagcaactggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgatcgtgtgactgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaggtggagagaagaattaggtgagcagaagaaggaattcccactgagttttaagacatttggggatgcaatccctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaatgggaagataggttctataaagctaatcgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattgatcggtttaatgacggttggaaccatttaaagagggtaagctatattactgtatgtatattagtatgttcctggataatttagaagcttttgtctgttgtcttttgcagtttatgaagttagtttgctgttgtcatgttacttgttactttaaaaagctttttgtagtttttgagcaactagtatggaatgctcttcctgtattgcttggaaaattcacaaaagtggtttttcggct

SEQ ID NO: 5 - DNA, 1998 bp, synthetic construct
  misc_feature 1..1998: note = cDNA of wildtype BvALS
  source 1..1998: mol_type = other DNA
atggcggctaccttcacaaacccaacattttccccttcctcaactcaattaaccaaaaccctaaaatcccaatcttccatttcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggtgcatctatggaaatccaccaagctctgacgcgctctaaaaccatccgcaatgtcctcccccgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccaattgttgaggtaacaaggtctattactaagcataattatttggttttggatgtagaagatattcctagaattgttaaggaagccttttttttagctaattctggcaggcctggacctgttttgattgatcttcctaaagatattcagcagcaactggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgatcgtgtgactgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaggtggagagaagaattaggtgagcagaagaaggaattcccactgagttttaagacatttggggatgcaatccctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaatgggaagataggttctataaagctaatcgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattga

SEQ ID NO: 6 - protein, 665 aa, Beta vulgaris
MAATFTNPTFSPSSTQLTKTLKSQSSISSTLPFSTPPKTPTPLFHRPLQISSSQSHKSSAIKTQTQAPSSPAIEDSSFVSRFGPDEPRKGSDVLVEALEREGVTNVFAYPGGASMEIHQALTRSKTIRNVLPRHEQGGVFAAEGYARATGKVGVCIATSGPGATNLVSGLADALLDSVPLVAITGQVPRRMIGTDAFQETPIVEVTRSITKHNYLVLDVEDIPRIVKEAFFLANSGRPGPVLIDLPKDIQQQLVVPDWDRPFKLGGYMSRLPKSKFSTNEVGLLEQIVRLMSESKKPVLYVGGGCLNSSEELRRFVELTGIPVASTLMGLGSYPCNDELSLHMLGMHGTVYANYAVDKADLLLAFGVRFDDRVTGKLEAFASRAKIVHIDIDSAEIGKNKQPHVSICADVKLALRGMNKILESRIGKLNLDFSRWREELGEQKKEFPLSFKTFGDAIPPQYAIQVLDELTNGNAIISTGVGQHQMWAAQHYKYRNPRQWLTSGGLGAMGFGLPAAIGAAVARPDAVVVDIDGDGSFIMNVQELATIRVENLPVKIMLLNNQHLGMVVQWEDRFYKANRAHTYLGNPSKSADIFPDMLKFAEACDIPSARVSNVADLRAAIQTMLDTPGPYLLDVIVPHQEHVLPMIPSGAGFKDTITEGDGRTSY

SEQ ID NO: 7 - DNA, 42 bp, synthetic construct
  misc_feature 1..42: note = marker sytxalss14as001 Primer_AlleleX
  source 1..42: mol_type = other DNA
gaaggtgaccaagttcatgctttcggggttaggtttgatgaa

SEQ ID NO: 8 - DNA, 45 bp, synthetic construct
  misc_feature 1..45: note = marker sytxalss14as001 Primer_AlleleY
  source 1..45: mol_type = other DNA
gaaggtcggagtcaacggattgctttcggggttaggtttgatgat

SEQ ID NO: 9 - DNA, 22 bp, synthetic construct
  misc_feature 1..22: note = marker sytxalss14as001 Primer_Common
  source 1..22: mol_type = other DNA
ggctagcaaacgcctcgagctt

SEQ ID NO: 10 - DNA, 2262 bp, synthetic construct
  misc_feature 1..2262: note = genomic DNA of mutant BvALS_W569L
  source 1..2262: mol_type = other DNA
tctctctccaaaactccaaacaacaacaatggcggctaccttcacaaacccaacattttccccttcctcaactccattaaccaaaaccctaaaatcccaatcttccatctcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggtgcatctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccaattgttgaggtgacaaggtctattactaagcataattatttagttttggatgtagaggatattcctagaattgttaaggaagccttttttttagctaattctggtaggcctggacctgttttgattgatcttcctaaagatattcagcagcaattggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgatcgtgtgaccgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaagtggagagaagaattaggtgagcagaagaaggaattcccactgagttttaagacatttggggatgcaattcctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaattggaagataggttctataaagctaaccgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattgatcggtttaatgacggttggaaccatttaaagagggtaagctatattactgtatgtatattagtatgttcctggataatttagaagcttttgtctgttgtcttttgcagtttatgaagttagtttgctgttgtcatgttacttgttactttaaaaagctttttgtagtttttgagcaactagtatggaatgctcttcctgtattgcttggaaaattcacaaaagtggtttttcggct

SEQ ID NO: 11 - DNA, 1998 bp, synthetic construct
  misc_feature 1..1998: note = cDNA of mutant BvALS_W569L
  source 1..1998: mol_type = other DNA
atggcggctaccttcacaaacccaacattttccccttcctcaactccattaaccaaaaccctaaaatcccaatcttccatctcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggtgcatctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccaattgttgaggtgacaaggtctattactaagcataattatttagttttggatgtagaggatattcctagaattgttaaggaagccttttttttagctaattctggtaggcctggacctgttttgattgatcttcctaaagatattcagcagcaattggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgatcgtgtgaccgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaagtggagagaagaattaggtgagcagaagaaggaattcccactgagttttaagacatttggggatgcaattcctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaattggaagataggttctataaagctaaccgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattga

SEQ ID NO: 12 - protein, 665 aa, synthetic construct
  REGION 1..665: note = protein of mutant BvALS_W569L
MAATFTNPTFSPSSTPLTKTLKSQSSISSTLPFSTPPKTPTPLFHRPLQISSSQSHKSSAIKTQTQAPSSPAIEDSSFVSRFGPDEPRKGSDVLVEALEREGVTNVFAYPGGASMEIHQALTRSKTIRNVLPRHEQGGVFAAEGYARATGKVGVCIATSGPGATNLVSGLADALLDSVPLVAITGQVPRRMIGTDAFQETPIVEVTRSITKHNYLVLDVEDIPRIVKEAFFLANSGRPGPVLIDLPKDIQQQLVVPDWDRPFKLGGYMSRLPKSKFSTNEVGLLEQIVRLMSESKKPVLYVGGGCLNSSEELRRFVELTGIPVASTLMGLGSYPCNDELSLHMLGMHGTVYANYAVDKADLLLAFGVRFDDRVTGKLEAFASRAKIVHIDIDSAEIGKNKQPHVSICADVKLALRGMNKILESRIGKLNLDFSKWREELGEQKKEFPLSFKTFGDAIPPQYAIQVLDELTNGNAIISTGVGQHQMWAAQHYKYRNPRQWLTSGGLGAMGFGLPAAIGAAVARPDAVVVDIDGDGSFIMNVQELATIRVENLPVKIMLLNNQHLGMVVQLEDRFYKANRAHTYLGNPSKSADIFPDMLKFAEACDIPSARVSNVADLRAAIQTMLDTPGPYLLDVIVPHQEHVLPMIPSGAGFKDTITEGDGRTSY